SEQUENCE LISTING (104 sequences)

SEQ ID NO: 1 - DNA, 651 bp, Artificial Sequence
  DNA encoding CD16A_V158_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 2 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQDYKDDDDK

SEQ ID NO: 3 - DNA, 651 bp, Artificial Sequence
  DNA encoding CD16A_V158_K120D_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacgatgtgacctacctgcagaacggcaagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 4 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSKNVSSETVNITITQGLAVSTISSFFPPGYQDYKDDDDK

SEQ ID NO: 5 - DNA, 651 bp, Artificial Sequence
  DNA encoding CD16A_V158_K120E_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacgaggtgacctacctgcagaacggcaagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 6 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSKNVSSETVNITITQGLAVSTISSFFPPGYQDYKDDDDK

SEQ ID NO: 7 - DNA, 651 bp, Artificial Sequence
  DNA encoding CD16A_V158_K128D_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgatggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 8 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGDGRKYFHHNSDFYIPKATLKDSGSYFCRGLVGSKNVSSETVNITITQGLAVSTISSFFPPGYQDYKDDDDK

SEQ ID NO: 9 - DNA, 651 bp, Artificial Sequence
  DNA encoding CD16A_V158_K128E_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 10 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGRKYFHHNSDFYIPKATLKDSGSYFCRGLVGSKNVSSETVNITITQGLAVSTISSFFPPGYQDYKDDDDK

SEQ ID NO: 11 - DNA, 651 bp, Artificial Sequence
  DNA encoding CD16A_V158_K131D_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 12 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQDYKDDDDK

SEQ ID NO: 13 - DNA, 651 bp, Artificial Sequence
  DNA encoding CD16A_V158_K131E_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 14 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGREYFHHNSDFYIPKATLKDSGSYFCRGLVGSKNVSSETVNITITQGLAVSTISSFFPPGYQDYKDDDDK

SEQ ID NO: 15 - DNA, 651 bp, Artificial Sequence
  DNA encoding CD16A_V158_K161D_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagaaagtacttccaccacaactccgacttctacatccccaaggctaccctgaaggactccggctcctacttctgtagaggcctcgtgggctccgataacgtgtcctccgagacagtgaacatcaccatcacacagggcctcgccgtgtctaccatctccagctttttcccacctggctaccaggactacaaggacgacgacgacaagtga

SEQ ID NO: 16 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSDNVSSETVNITITQGLAVSTISSFFPPGYQDYKDDDDK

SEQ ID NO: 17 - DNA, 651 bp, Artificial Sequence
  DNA encoding CD16A_V158_K161E_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagaaagtacttccaccacaactccgacttctacatccccaaggctaccctgaaggactccggctcctacttctgtagaggcctcgtgggctccgagaacgtgtcctccgagacagtgaacatcaccatcacacagggcctcgccgtgtctaccatctccagctttttcccacctggctaccaggactacaaggacgacgacgacaagtga

SEQ ID NO: 18 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSENVSSETVNITITQGLAVSTISSFFPPGYQDYKDDDDK

SEQ ID NO: 19 - DNA, 57 bp, Artificial Sequence
  Signal sequence
  CDS (1)..(57)
atggaatttggcctgtcctgggtgttcctggtggccattctgaaaggcgtgcagtgc

SEQ ID NO: 20 - protein, 19 aa, Artificial Sequence
  Synthetic Construct
MEFGLSWVFLVAILKGVQC

SEQ ID NO: 21 - DNA, 360 bp, Artificial Sequence
  DNA encoding Trastuzumab_VH_1
  CDS (1)..(360)
gaggtgcagctggttgaatctggcggaggattggttcagcctggcggctctctgagactgtcttgtgccgcctctggcttcaacatcaaggacacctacatccactgggtccgacaggcccctggcaaaggattggaatgggtcgccagaatctaccccaccaacggctacaccagatacgccgactctgtgaagggcagattcaccatctccgccgacacctccaagaacaccgcctacctgcagatgaactccctgagagccgaggacaccgccgtgtactactgctctagatggggaggcgacggcttctacgccatggattattggggccagggcaccctggtcaccgttagttct

SEQ ID NO: 22 - protein, 120 aa, Artificial Sequence [sequence omitted]

SEQ ID NO: 23 - DNA, 993 bp, Artificial Sequence
  DNA encoding Fc_wt (CH_IgG1)
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 24 - protein, 330 aa, Artificial Sequence [sequence omitted]

SEQ ID NO: 25 - DNA, 66 bp, Artificial Sequence
  Signal sequence
  CDS (1)..(66)
atggatatgagagtgcccgctcagctgctgggactgctgctgttgtggttgagaggcgccagatgc

SEQ ID NO: 26 - protein, 22 aa, Artificial Sequence
  Synthetic Construct
MDMRVPAQLLGLLLLWLRGARC

SEQ ID NO: 27 - DNA, 645 bp, Artificial Sequence
  DNA encoding Trastuzumab_Light Chain
  CDS (1)..(645)
gacatccagatgacccagtctccatcctctctgtccgcctctgtgggcgacagagtgaccatcacctgtagagccagccaggacgtgaacaccgccgtggcttggtatcagcagaagcctggcaaggcccctaagctgctgatctactccgcctccttcctgtactctggcgtgccctctagattctccggctctagatctggcaccgactttaccctgacaatctccagcctgcagcctgaggacttcgccacctactactgccagcagcactacaccacacctccaacctttggccagggcaccaaggtggaaatcaagcgtacggtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 28 - protein, 214 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 993 bp, Artificial Sequence
  DNA encoding Fc_E294K
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagagaagcagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 30 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREKQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 993 bp, Artificial Sequence
  DNA encoding Fc_E294R
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagagagacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 32 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPRERQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 993 bp, Artificial Sequence
  DNA encoding Fc_S239K
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggacctaaggtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 34 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 993 bp, Artificial Sequence
  DNA encoding Fc_S239R
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggacctagagtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 36 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPRVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 57 bp, Artificial Sequence
  Signal sequence
  CDS (1)..(57)
atggaatggtcctgggtgttcctgttcttcctgtccgtgaccaccggcgtgcactct

SEQ ID NO: 38 - protein, 19 aa, Artificial Sequence
  Synthetic Construct
MEWSWVFLFFLSVTTGVHS

SEQ ID NO: 39 - DNA, 360 bp, Artificial Sequence
  DNA encoding Trastuzumab_VH_2
  CDS (1)..(360)
gaagtgcagctggttgaatctggcggcggattggttcagcctggcggatctctgagactgtcttgtgccgcctccggcttcaacatcaaggacacctacatccactgggtccgacaggcccctggcaaaggattggaatgggtcgccagaatctaccccaccaacggctacaccagatacgccgactctgtgaagggcagattcaccatctccgccgacacctccaagaacaccgcctacctgcagatgaactccctgagagccgaggacaccgccgtgtactactgctctagatggggaggcgacggcttctacgccatggattattggggccagggcaccctggtcaccgtttcttcc

SEQ ID NO: 40 - protein, 120 aa, Artificial Sequence [sequence omitted]

SEQ ID NO: 41 - DNA, 993 bp, Artificial Sequence
  DNA encoding Fc_D265K
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtgaaggtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 42 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVKVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 993 bp, Artificial Sequence
  DNA encoding Fc_D265R
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtgagagtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 44 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVRVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 993 bp, Artificial Sequence
  DNA encoding Fc_E269K
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacaaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 46 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHKDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 993 bp, Artificial Sequence
  DNA encoding Fc_E269R
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacagagacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 48 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 651 bp, Artificial Sequence
  DNA encoding CD16A_V158_K128E_K131D_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 50 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGRDYFHHNSDFYIPKATLKDSGSYFCRGLVGSKNVSSETVNITITQGLAVSTISSFFPPGYQDYKDDDDK

SEQ ID NO: 51 - DNA, 651 bp, Artificial Sequence
  DNA encoding CD16A_V158_K128E_K131E_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgagggcagagagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 52 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGREYFHHNSDFYIPKATLKDSGSYFCRGLVGSKNVSSETVNITITQGLAVSTISSFFPPGYQDYKDDDDK

SEQ ID NO: 53 - DNA, 651 bp, Artificial Sequence
  DNA encoding CD16A_V158_K131D_N38Q_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggaccagtctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 54 - protein, 216 aa, Artificial Sequence
  Synthetic Construct
MWQLLLPTALLLLVSAGMRTEDLPKAVVFLEPQWYRVLEKDSVTLKCQGAYSPEDQSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQDYKDDDDK

SEQ ID NO: 55 - DNA, 651 bp, Artificial Sequence
  DNA encoding CD16A_V158_K131D_N74Q_FLAG
  CDS (1)..(651)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagacccagctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggactacaaggacgacgacgacaagtga

SEQ ID NO: 56 - protein, 216 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTQLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQDYKDDDDK

SEQ ID NO: 57 - DNA, 993 bp, Artificial Sequence
  DNA encoding Fc_E269R_E294K
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacagagacccagaagtgaagttcaattggtacgtggacggcgtggaagtgcacaacgccaagaccaagcctagagagaagcagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 58 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHRDPEVKFNWYVDGVEVHNAKTKPREKQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 993 bp, Artificial Sequence
  DNA encoding Fc_E269R_E294R
  CDS (1)..(993)
gctagcaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacagagacccagaagtgaagttcaattggtacgtggacggcgtggaagtgcacaacgccaagaccaagcctagagagagacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccttccgatatcgccgtggaatgggagtctaacggccagccagagaacaactacaagacaacccctcctgtgctggactccgacggctcattcttcctgtactccaagctgacagtggataagtcccggtggcagcagggcaacgtgttctcttgctctgtgatgcacgaggccctgcacaaccactacacacagaagtctctgtctctgagccccggcaagtga

SEQ ID NO: 60 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHRDPEVKFNWYVDGVEVHNAKTKPRERQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 57 bp, Artificial Sequence
  Signal sequence
  CDS (1)..(57)
atggaatttggcctgtcctgggtgttcctggtggctctgttgagaggcgtgcagtgt

SEQ ID NO: 62 - protein, 19 aa, Artificial Sequence
  Synthetic Construct
MEFGLSWVFLVALLRGVQC

SEQ ID NO: 63 - DNA, 357 bp, Artificial Sequence
  DNA encoding Cetuximab VH
  CDS (1)..(357)
caggtgcagctgaagcagtctggacctggactggtgcagccttctcagtccctgtccatcacctgtaccgtgtccggcttctccctgaccaattacggcgtgcactgggtccgacagtctccaggcaaaggactggaatggctgggagtgatttggagcggcggcaacaccgactacaacacccctttcacctctcggctgagcatcaacaaggacaactccaagagccaggtgttcttcaagatgaactccctgcagtccaacgacaccgccatctactactgcgctagagccctgacctactacgactacgagttcgcctattggggccagggcacactggtcacagtttccgct

SEQ ID NO: 64 - protein, 119 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 65 - DNA, 57 bp, Artificial Sequence
  Signal sequence
  CDS (1)..(57)
atgttgccttctcagctgatcggcttcctgctgctgtgggtgccagcttctagaggc

SEQ ID NO: 66 - protein, 19 aa, Artificial Sequence
  Synthetic Construct
MLPSQLIGFLLLWVPASRG

SEQ ID NO: 67 - DNA, 645 bp, Artificial Sequence
  DNA encoding Cetuximab_Light Chain
  CDS (1)..(645)
gacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggcgctggcaccaagctggaactgaagcgtacggtggctgccccttccgtgttcatcttcccaccttccgacgaacagctgaagtccggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgagcaaggccgactacgagaagcacaaggtgtacgcctgcgaagtgacccatcagggcctgtctagccctgtgaccaagtctttcaaccggggcgagtgctga

SEQ ID NO: 68 - protein, 214 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 57 bp, Artificial Sequence
  Signal sequence
  CDS (1)..(57)
atggaatggtcttgggtgttcctgttcttcctgtccgtgaccaccggcgtgcactct

SEQ ID NO: 70 - protein, 19 aa, Artificial Sequence
  Synthetic Construct
MEWSWVFLFFLSVTTGVHS

SEQ ID NO: 71 - DNA, 348 bp, Artificial Sequence
  DNA encoding Edrecolomab_VH
  CDS (1)..(348)
caggttcagttgcagcagtctggcgccgaactcgtcagacctggcacatctgtgaaggtgtcctgcaaggcctctggctacgccttcaccaactacctgatcgagtgggtcaagcagaggcctggacagggactcgagtggatcggcgttatcaatcctggctctggcggcaccaattacaacgagaagttcaagggcaaagctaccctgaccgccgacaagtcctcttccaccgcttacatgcagctgtccagcctgacctctgacgactccgccgtgtacttctgtgccagagatggcccttggtttgcctattggggccagggcacactggtcaccgtttctgct

SEQ ID NO: 72 - protein, 116 aa, Artificial Sequence
  Synthetic Construct
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDGPWFAYWGQGTLVTVSA

SEQ ID NO: 73 - DNA, 60 bp, Artificial Sequence
  Signal sequence
  CDS (1)..(60)
atgtctgtgcctacacaggttctgggactgctgctgctgtggctgaccgacgccagatgc

SEQ ID NO: 74 - protein, 20 aa, Artificial Sequence
  Synthetic Construct
MSVPTQVLGLLLLWLTDARC

SEQ ID NO: 75 - DNA, 645 bp, Artificial Sequence
  DNA encoding edrecolomab_Light Chain
  CDS (1)..(645)
aacatcgtgatgacccagtctccaaagtccatgtccatgagcgtgggcgagagagtgaccctgacctgcaaggcctctgagaacgtggtcacctacgtgtcctggtatcagcagaagcccgagcagtcccctaagctgctgatctacggcgcctctaacagatacaccggcgtgcccgatagattcaccggctctggctctgccaccgactttaccctgaccatcagctctgtgcaggccgaggatctggccgattaccattgtggccagggctacagctacccctacacatttggcggaggcaccaagctggaaatcaagcgtacggtggccgctccttccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 76 - protein, 214 aa, Artificial Sequence
  Synthetic Construct
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 711 bp, Homo sapiens
  CDS (1)..(711)
agaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 78 - protein, 236 aa, Homo sapiens
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 79 - DNA, 765 bp, Artificial Sequence
  DNA encoding CD16A_V158_K131D Full
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 80 - protein, 254 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 81 - DNA, 765 bp, Artificial Sequence
  DNA encoding CD16A_V158_K128E_K131D Full
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 82 - protein, 254 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGRDYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 83 - DNA, 765 bp, Artificial Sequence
  DNA encoding CD16A_V158_K128E_K131E Full
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcgagggcagagagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 84 - protein, 254 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGREYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 85 - DNA, 765 bp, Artificial Sequence
  DNA encoding CD16A_V158_K131D_N38Q Full
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggaccagtctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 86 - protein, 254 aa, Artificial Sequence
  Synthetic Construct
MWQLLLPTALLLLVSAGMRTEDLPKAVVFLEPQWYRVLEKDSVTLKCQGAYSPEDQSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 87 - DNA, 765 bp, Artificial Sequence
  DNA encoding CD16A_V158_K131D_N74Q Full
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagacccagctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagagattacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 88 - protein, 254 aa, Artificial Sequence
  Synthetic Construct
MWQLLLPTALLLLVSAGMRTEDLPKAVVFLEPQWYRVLEKDSVTLKCQGAYSPEDNSTQWFHNESLISSQASSYFIDAATVDDSGEYRCQTQLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 89 - DNA, 765 bp, Homo sapiens
  CDS (1)..(765)
atgtggcagctgctgctgcctacagctctgctgctgttggtgtctgccggcatgagaaccgaggatctgcctaaggccgtggtgttcctggaacctcagtggtacagagtgctggaaaaggacagcgtgaccctgaagtgccagggcgcttactctcccgaggacaactctacccagtggttccacaacgagtccctgatctccagccaggcctcctcctactttatcgatgccgccaccgtggacgactctggcgagtatagatgccagaccaacctgagcaccctgagcgatcctgtgcagctggaagtgcatatcggctggctgttgctgcaggcccctagatgggtgttcaaagaagaggaccccatccacctgagatgccactcttggaagaacaccgctctgcacaaagtgacctacctgcagaacggcaagggcagaaagtacttccaccacaactccgacttctacatccccaaggctaccctgaaggactccggctcctacttctgtagaggcctcgtgggctccaagaacgtgtcctccgagacagtgaacatcaccatcacacagggcctcgccgtgtctaccatctccagctttttcccacctggctaccaggtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 90 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 91 - protein, 8 aa, Artificial Sequence
  FLAG
DYKDDDDK

SEQ ID NO: 92 - DNA, 1311 bp, Artificial Sequence
  CD16A_V158_8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagagaggccgcaagaagctgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 93 - protein, 436 aa, Artificial Sequence
  Synthetic Construct
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 1311 bp, Artificial Sequence
  CD16A_V158_K131D 8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagagactacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagcggggcagaaagaaactgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 95 - protein, 436 aa, Artificial Sequence
  Synthetic Construct
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 1311 bp, Artificial Sequence
  CD16A_V158_K128E_K131D 8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcgagggcagagactacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagcggggcagaaagaaactgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 97 - protein, 436 aa, Artificial Sequence
  Synthetic Construct
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGRDYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - DNA, 1311 bp, Artificial Sequence
  CD16A_V158_K128E_K131E 8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcgagggcagagagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagcggggcagaaagaaactgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 99 - protein, 436 aa, Artificial Sequence
  Synthetic Construct
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGEGREYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1311 bp, Artificial Sequence
  CD16A_V158_K131D_N38Q 8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggaccagagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagagactacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagcggggcagaaagaaactgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 101 - protein, 436 aa, Artificial Sequence
  Synthetic Construct
MALPVTALLLPLALLLHAARPGMRTEDLPKAVVFLEPQWYRVLEKDSVTLKCQGAYSPEDQSTQWFHNESLISSQASSYFIDAATVDDSGEYRCQTNLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1311 bp, Artificial Sequence
  CD16A_V158_K131D_N74Q 8BBz
  CDS (1)..(1311)
atggctctgcctgttacagctctgctgctgcctctggctctgcttctgcatgctgccagacctggcatgagaaccgaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgtcaaacccagctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagagactacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctaccagaccaccacaccagctcctagacctccaactcctgctccaacaatcgccagccagcctctgtctctgaggccagaagcttgtagacctgctgctggcggagccgtgcatacaagaggactggatttcgcctgcgatatctacatctgggcccctctggctggcacatgtggcgttctgctgctcagcctggtcatcaccctgtactgcaagcggggcagaaagaaactgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgcctccaagataa

SEQ ID NO: 103 - protein, 436 aa, Artificial Sequence
  Synthetic Construct
MALPVTALLLPLALLLHAARPGMRTEDLPKAVVFLEPQWYRVLEKDSVTLKCQGAYSPEDNSTQWFHNESLISSQASSYFIDAATVDDSGEYRCQTQLSTLSDPVQLEVHIGWLLLQAPRWVFKEEDPIHLRCHSWKNTALHKVTYLQNGKGRDYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 21 aa, Artificial Sequence
  signal sequence
MALPVTALLLPLALLLHAARP

(19) United States
(12) Patent Application Publication
(10) Pub. No.: US 2023/0303658 A1
(43) Pub. Date: Sep. 28, 2023

(54) HUMAN NON-NATURALLY OCCURRING MODIFIED FC REGION OF IGG SPECIFICALLY BINDING TO NON-NATURALLY OCCURRING MODIFIED FC RECEPTOR

(71) Applicant: Astellas Pharma Inc., Tokyo (JP)

(72) Inventors: Ryuichi Moriya, Tokyo (JP); Hiroki Shirai, Tokyo (JP); Shinji Soga, Tokyo (JP); Naoko Shimada, Tokyo (JP); David William Russell, Seattle, WA (US)

(21) Appl. No.: 18/021,581

(22) PCT Filed: Aug. 18, 2021
(86) PCT No.: PCT/IB2021/000556

§ 371 Date: Feb. 16, 2023

Related U.S. Application Data
(60) Provisional application No. 63/067,629, filed on Aug. 19, 2020.

(51) Int. Cl. C07K 14/735; C07K 16/30; A61P 35/00; C12N 5/0735

(52) U.S. Cl. CPC C07K 14/70535; C07K 16/30; A61P 35/00; C12N 5/0606; C12N 2510/00

(57) ABSTRACT

The present invention provides a polypeptide comprising a modified Fc region of an IgG, and a modified Fcy receptor that binds specifically to the polypeptide, and methods for treating or preventing a disease or a disorder in a patient using immunotherapy.

### RELATED APPLICATIONS

This application claims the benefit of priority to U.S. Provisional Application No. 63/067,629, filed on Aug. 19, 2020. The entire contents of the foregoing application are incorporated herein by reference.

### BACKGROUND OF THE INVENTION

Research and development of cancer immunotherapies that eliminate cancer cells by utilizing the immune system have advanced in recent years (Nature Reviews Drug Discovery (2019) 18, pp. 899-900). In particular, chimeric antigen receptor T-cells (CAR-T) expressing a chimeric antigen receptor (CAR) in which an antigen recognition site and an activation signal transduction site are linked are reported to have a dramatic therapeutic effect after a single administration (The New England Journal of Medicine (2017) 377, pp. 2545-2554), and a new cancer treatment method is widely anticipated.

Several problems have arisen in the development of conventional CAR-T therapies for various cancer types (Nature Reviews Clinical Oncology (2020) 17, pp. 147-167). First, there is a problem with safety as serious immune response-based side effects such as cytokine release syndrome have been reported in immune cell therapy using CAR-T. In conventional CAR-T, it is difficult to address the occurrence of side effects because the activity of CAR-expressing cells cannot be selectively regulated after the cells have been transferred to a patient. Cancer tissue has a mechanism for acquiring treatment resistance, and problems such as the appearance of cancer cells that have lost cancer-related antigens over the course of treatment (Cancer Discovery (2018) 8 (10), pp. 1219-1226) and cell populations with diverse properties (heterogeneity) have also arisen. Current treatments using conventional CAR-T, which target a single antigen, cannot overcome these problems.

In order to solve these problems, combination therapies using cancer target molecules and immune effector cells such as T cells and natural killer (NK) cells are being studied. Antibody molecules with tag molecules such as FITC and CAR-T that recognize these tag molecules have been developed (WO 2012/082841, WO 2016/030414, and WO 2017/091546). By combining various cancer target molecules with tag-recognizing CAR-T, this technique can impart cytotoxic activity against multiple cancer antigens and cancer cells to a single type of tag-recognizing CAR-T. However, there is a risk that the immunogenicity of the antibody molecule will be enhanced by imparting a tag molecule that does not originally exist in vivo to the antibody.

Meanwhile, a technique has also been developed that utilizes an effector cell and the antibody molecule itself with no added tag molecule. The antibody is able to bind to the Fc receptor via the Fc region and transmit a signal to the effector cell. Cells that have been created for use with an antibody having a cancer antigen recognition function include NK cells that express an Fc fragment of IgG receptor IIIa (known as FcγRIIIA, CD16A) as an Fc receptor (JCI Insight. (2019) 4 (20): e130688) and T cells (CD16A CAR-T) or NK cells (CD16A CAR-NK) expressing a CAR fused with CD16A and a signal transduction site (British Journal of Cancer (2019) 120 (1), pp. 79-87, Oncotarget. (2017) 8 (23), pp. 37128-37139). Because a single type of CD16A-expressing NK and T cell can be combined with various cancer targeting antibodies, these cells also have the potential to become an excellent therapeutic method that is capable of acquiring cytotoxic activity against cells expressing various antigens. Because the antibody molecules themselves are used, it is believed that a therapeutic method can be obtained that has lower immunogenicity and a higher level of safety than those using tagged antibodies.

However, there is a large amount of endogenous immunoglobulins present in vivo in serum, and these also bind to CD16A. The presence of soluble Fc receptors in serum has also been confirmed (Journal of Clinical Investigation (1990) 86, pp. 416-423), and these bind to therapeutic antibodies. In other words, when CD16A on effector cells is occupied by immunoglobulin in the body or an administered antibody is occupied by soluble Fc receptors, the administered antibody is unable to transmit an activation signal to the effector cells, and diminished drug efficacy is anticipated. Also, when CD16A-expressing NK cells, CD16A CAR-T or CAR-NK cells are administered to a patient with antibody molecules that recognize the patient's own tissue, such as autoantibodies, they may be activated against the patient's own tissue and cause tissue damage. CD16A mutants are known that bind to afucosylated antibodies but not to non-afucosylated endogenous immunoglobulins (WO 2017/161333), but these afucosylated antibodies transmit signals not only to CD16A mutants but also to endogenous CD16A. Combinations of CD16A mutants that do not bind to endogenous immunoglobulin and Fc mutants that do not bind to endogenous CD16A, and combinations of mutants that combine specifically with each other are unknown to date.

### SUMMARY OF THE INVENTION

The present invention relates to a polypeptide comprising a modified Fc region and a modified Fcγ receptor that binds specifically to this polypeptide that can be used as an immunotherapy. It is an object of the present invention to provide an immunotherapy in which endogenous molecules do not diminish drug efficacy.

The present invention is based, at least in part, on the discovery of combinations of non-naturally occurring Fcγ receptor mutants that do not bind to endogenous immunoglobulins and non-naturally occurring Fc region mutants that do not bind to endogenous Fcγ receptors, the use of these combinations to specifically bind the non-naturally occurring Fcγ receptor mutants and non-naturally occurring Fc region mutants as an immunotherapy for treating a subject, and the methods for making these combinations. For example, the present inventors extracted the amino acid site that affects the binding activity between CD16A and the Fc region of an antibody in silico, prepared mutants in which mutations were introduced to CD16A and the Fc region of the antibody, and evaluated the change in binding activity to the wild type, discovering that binding activity to the wild type had decreased (Examples 1-4). Based on this discovery, the present inventors identified non-naturally occurring Fc region mutants showing no binding activity to wild type CD16A, but maintaining high binding activity to non-naturally occurring mutated CD16A. The inventors also identified combinations of non-naturally occurring modified Fc regions that did not bind to wild type CD16A and non-naturally occurring mutated CD16A that did not bind to the wild type antibody Fc region. Because similar results were obtained using a plurality of antibodies against different antigens, it was also determined that these characteristics do not depend on the antigen (Examples 5-8). The present inventors further established natural killer (NK) cell lines expressing wild type CD16A or non-naturally occurring mutated CD16A, and confirmed that the antibody-dependent cellular cytotoxicity (ADCC) reflects the binding activity confirmed in Example 6 (Examples 10, 11). Furthermore, the present inventors confirmed the binding activity characteristics in Example 6 in the presence of excess IgG1 antibodies (Example 9).

The present invention provides the following aspects as compositions and methods expected to be useful in medicine and industry.

In one aspect, the present invention provides a polypeptide comprising a modified Fc region of IgG, wherein the modified Fc region is non-naturally occurring and comprises at least one amino acid mutation compared to an Fc region of a wild type or naturally occurring IgG. The polypeptide has essentially no binding activity to a wild type or naturally occurring Fcγ receptor and is capable of binding to a non-naturally occurring Fcγ receptor comprising at least one amino acid mutation compared to the wild type or naturally occurring Fcγ receptor.

In some embodiments, the wild type or naturally occurring Fcγ receptor is a wild type or naturally occurring CD16A, and the non-naturally occurring Fcγ receptor comprising at least one amino acid mutation is a non-naturally occurring CD16A comprising at least one amino acid mutation.

In some embodiments, the wild type or naturally occurring CD16A comprises the amino acid sequence shown in SEQ ID NO: 78.

In some embodiments, the CD16A comprising at least one amino acid mutation comprises at least one mutation selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K131E mutation).

In some embodiments, the CD16A comprising at least one amino acid mutation comprises one or both of the K131D mutation and the K128E mutation. In other embodiments, the CD16A comprising at least one amino acid mutation comprises one or both of the K131E mutation and the K128E mutation. In some embodiments, the CD16A comprising at least one amino acid mutation comprises the K131D mutation, and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation). In other embodiments, the CD16A comprising at least one amino acid mutation comprises the amino acid sequence shown in SEQ ID NO: 80, SEQ ID NO: 82, SEQ ID NO: 84, SEQ ID NO: 86, or SEQ ID NO: 88.

In some embodiments, the polypeptide comprises a modified Fc region of human Igγ1, and the modified Fc region comprises (i) a mutation from a glutamic acid to an arginine at a position corresponding to position 269 according to EU index numbering (E269R mutation) and (ii) at least one mutation selected from (a) a glutamic acid to an arginine at a position corresponding to position 294 according to EU index numbering (E294R mutation) and (b) a glutamic acid to a lysine at a position corresponding to position 294 according to EU index numbering (E294K mutation).

In some embodiments, the polypeptide is an antibody. In other embodiments, the polypeptide is an antibody that binds to a cancer antigen.

In another aspect, the present invention provides a method of treating or preventing a disease or a disorder in a patient using immunotherapy. The method comprises administering to a patient a polypeptide as described herein and a cell expressing the non-naturally occurring Fcγ receptor comprising at least one amino acid mutation compared to a wild type or naturally occurring Fcγ receptor, wherein the polypeptide is capable of binding to said non-naturally occurring Fcγ receptor comprising at least one amino acid mutation.

In some embodiments, the cell is a human immune cell. In other embodiments, the human immune cell is a cell selected from a T cell, macrophage, dendritic cell, NKT-cell. NK cell, microglia, osteoclast, granulocyte, monocyte, and innate immune cell. In some embodiments, the cell is derived from a stem cell. In other embodiments, the stem cell is selected from a pluripotent stem cell, hematopoietic stem cell, adult stem cell, fetal stem cell, mesenchymal stem cell, postpartum stem cell, multipotent stem cell, and embryonic germ cell. In some embodiments, the stem cell is a pluripotent stem cell. In other embodiments, the pluripotent stem cell is an induced pluripotent stem cell (iPS cell) or an embryonic stem cell (ES cell).

In some embodiments, the cell comprises a genetically engineered disruption in a beta-2 microglobulin (B2M) gene.

In some embodiments, the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain. In some embodiments, the HLA-1α chain is selected from HLA-A, HLA-B, HLA-C, HLA-E, HLA-F, and HLA-G.

In some embodiments, the cell comprises a genetically engineered disruption in a human leukocyte antigen (HLA) class II-related gene. In some embodiments, the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

In some embodiments, the cell comprises one or more polynucleotides encoding a single chain fusion HLA class II protein or an HLA class II protein.

In some embodiments, the method is a method for treating or preventing cancer.

In one aspect, the present disclosure provides a pharmaceutical composition comprising a polypeptide as described herein and a pharmaceutically acceptable excipient.

In some embodiments, the pharmaceutical composition is for combined use with a cell for immunotherapy, wherein the cell expresses a non-naturally occurring Fcγ receptor comprising at least one amino acid mutation compared to a wild type or naturally occurring Fcγ receptor, wherein the polypeptide is capable of binding to the non-naturally occurring Fcγ receptor comprising at least one amino acid mutation.

In some embodiments, the cell is a human immune cell. In some embodiments, the human immune cell is a cell selected from a T cell, macrophage, dendritic cell, NKT-cell, NK cell, microglia, osteoclast, granulocyte, monocyte, and innate immune cell. In some embodiments, the cell is derived from a stem cell. In some embodiments, the stem cell is selected from a pluripotent stem cell, hematopoietic stem cell, adult stem cell, fetal stem cell, mesenchymal stem cell, postpartum stem cell, multipotent stem cell, and embryonic germ cell. In some embodiments, the stem cell is a pluripotent stem cell. In some embodiments, the pluripotent stem cell is an induced pluripotent stem cell (iPS cell) or an embryonic stem cell (ES cell).

In some embodiments, the cell comprises a genetically engineered disruption in a beta-2 microglobulin (B2M) gene.

In some embodiments, the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain. In some embodiments, the HLA-1α chain is selected from HLA-A, HLA-B, HLA-C, HLA-E, HLA-F, and HLA-G.

In some embodiments, the cell comprises a genetically engineered disruption in a human leukocyte antigen (HLA) class II-related gene. In some embodiments, the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

In some embodiments, the cell comprises one or more polynucleotides encoding a single chain fusion HLA class II protein or an HLA class II protein.

In another aspect, the present invention provides a kit for treatment or prevention of a disease or disorder in a patient using immunotherapy. The kit comprises (i) a polypeptide as described herein and (ii) a cell expressing a non-naturally occurring Fcγ receptor comprising at least one amino acid mutation compared to a wild type or naturally occurring Fcγ receptor, wherein the polypeptide is capable of binding to the non-naturally occurring Fcγ receptor comprising at least one amino acid mutation.

In some embodiments, the cell is a human immune cell. In some embodiments, the human immune cell is a cell selected from a T cell, macrophage, dendritic cell, NKT-cell, NK cell, microglia, osteoclast, granulocyte, monocyte, and innate immune cell. In some embodiments, the cell is derived from a stem cell. In some embodiments, the stem cell is selected from a pluripotent stem cell, hematopoietic stem cell, adult stem cell, fetal stem cell, mesenchymal stem cell, postpartum stem cell, multipotent stem cell, and embryonic germ cell. In some embodiments, the stem cell is a pluripotent stem cell. In some embodiments, the pluripotent stem cell is an induced pluripotent stem cell (iPS cell) or an embryonic stem cell (ES cell).

In some embodiments, the cell comprises a genetically engineered disruption in a beta-2 microglobulin (B2M) gene.

In some embodiments, the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain. In some embodiments, the HLA-1α chain is selected from HLA-A, HLA-B, HLA-C, HLA-E, HLA-F, and HLA-G.

In some embodiments, the cell comprises a genetically engineered disruption in a human leukocyte antigen (HLA) class II-related gene. In some embodiments, the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

In some embodiments, the cell comprises one or more polynucleotides encoding a single chain fusion HLA class II protein or an HLA class II protein.

In one aspect, the present invention provides a cell expressing a non-naturally occurring CD16A comprising at least one amino acid mutation compared to a wild type or naturally occurring CD16A, wherein the at least one amino acid mutation is selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K131E mutation), and wherein the non-naturally occurring CD16A comprises an amino acid sequence having 90% or more amino acid sequence identity with SEQ ID NO: 78.

In some embodiments, the CD16A comprising at least one amino acid mutation comprises one or both of the K131D mutation and the K128E mutation.

In some embodiments, the CD16A comprising at least one amino acid mutation comprises one or both of the K131E mutation and the K128E mutation.

In some embodiments, the CD16A comprising at least one amino acid mutation comprises the K131D mutation, and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation).

In some embodiments, the CD16A comprising at least one amino acid mutation comprises the amino acid sequence shown in SEQ ID NO: 80, SEQ ID NO: 82, SEQ ID NO: 84, SEQ ID NO: 86, or SEQ ID NO: 88.

In some embodiments, the cell is a human immune cell. In some embodiments, the human immune cell is a cell selected from a T cell, macrophage, dendritic cell, NKT-cell, NK cell, microglia, osteoclast, granulocyte, monocyte, and innate immune cell. In some embodiments, the cell is derived from a stem cell. In some embodiments, the stem cell is selected from a pluripotent stem cell, hematopoietic stem cell, adult stem cell, fetal stem cell, mesenchymal stem cell, postpartum stem cell, multipotent stem cell, and embryonic germ cell. In some embodiments, the stem cell is a pluripotent stem cell. In some embodiments, the pluripotent stem cell is an induced pluripotent stem cell (iPS cell) or an embryonic stem cell (ES cell).

In some embodiments, the cell comprises a genetically engineered disruption in a beta-2 microglobulin (B2M) gene.

In some embodiments, the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain. In some embodiments, the HLA-1α chain is selected from HLA-A, HLA-B, HLA-C, HLA-E, HLA-F, and HLA-G.

In some embodiments, the cell comprises a genetically engineered disruption in a human leukocyte antigen (HLA) class II-related gene. In some embodiments, the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

In some embodiments, the cell comprises one or more polynucleotides encoding a single chain fusion HLA class II protein or an HLA class II protein.

In one aspect, the present invention provides a pharmaceutical composition comprising a cell as described herein and a pharmaceutically acceptable excipient.

In some embodiments, the pharmaceutical composition is for combined use with a polypeptide comprising a modified Fc region of IgG for immunotherapy, wherein the modified Fc region is non-naturally occurring and comprises at least one amino acid mutation compared to an Fc region of a wild type or naturally occurring IgG, and the polypeptide has essentially no binding activity to a wild type or a naturally occurring CD16A and is capable of binding to a non-naturally occurring CD16A comprising at least one amino acid mutation expressed by the cell.

In some embodiments, the polypeptide comprises a modified Fc region of human Igγ1, and the modified Fc region comprises (i) a mutation from a glutamic acid to an arginine at a position corresponding to position 269 according to EU index numbering (E269R mutation) and (ii) at least one mutation selected from (a) a glutamic acid to an arginine at a position corresponding to position 294 according to EU index numbering (E294R mutation) and (b) a glutamic acid to a lysine at a position corresponding to position 294 according to EU index numbering (E294K mutation).

In some embodiments, the polypeptide is an antibody. In some embodiments, the polypeptide is an antibody that binds to a cancer antigen.

In some embodiments, the pharmaceutical composition is for treating cancer.

In one aspect, the present invention provides a method for preparing a polypeptide containing a modified Fc region of IgG. The method comprises the steps of: 1) providing polypeptides comprising a modified Fc region of IgG, wherein the modified Fc region is non-naturally occurring and comprises at least one amino acid mutation compared to a wild type or naturally occurring IgG; 2) measuring the binding activity of the polypeptides obtained in 1) to a wild type or naturally occurring Fcγ receptor; 3) measuring the binding activity of the polypeptides obtained in 1) to a non-naturally occurring Fcγ receptor comprising at least one amino acid mutation compared to a wild type or naturally occurring Fcγ receptor; and 4) selecting from the polypeptides obtained in 1) a polypeptide having essentially no binding activity to the wild type or naturally occurring Fcγ receptor and which binds to the non-naturally occurring Fcγ receptor comprising at least one amino acid mutation.

In some embodiments, the wild type or naturally occurring Fcγ receptor is a wild type or naturally occurring CD16A and the non-naturally occurring Fcγ receptor comprising at least one amino acid mutation is a non-naturally occurring CD16A comprising at least one amino acid mutation.

In some embodiments, the wild type or naturally occurring CD16A comprises the amino acid sequence shown in SEQ ID NO: 78.

In some embodiments, the non-naturally occurring CD16A comprising at least one amino acid mutation comprises at least one mutation selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K131E mutation).

In some embodiments, the CD16A comprising at least one amino acid mutation comprises one or both of the K131D mutation and the K128E mutation.

In some embodiments, the CD16A comprising at least one amino acid mutation contains one or both of the K131E mutation and the K128E mutation.

In some embodiments, the CD16A comprising at least one amino acid mutation comprises the K131D mutation and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation).

In some embodiments, the non-naturally occurring polypeptide comprising a modified Fc region of IgG is an antibody. In some embodiments, the antibody is an antibody that binds to a cancer antigen.

In some embodiments, the polypeptide comprising a modified Fc region of IgG is an antibody, and the method further comprises a step of contacting the antibody with an immune cell expressing the non-naturally occurring Fcγ receptor comprising at least one amino acid mutation and a cell expressing an antigen to which the antibody binds, and measuring antibody-dependent cellular cytotoxicity (ADCC) activity.

In another aspect, the present invention provides a method for preparing a non-naturally occurring Fcγ receptor. The method comprises the steps of: 1) providing non-naturally occurring Fcγ receptors comprising at least one amino acid mutation compared with a wild type or naturally occurring Fcγ receptor; 2) providing a polypeptide comprising an Fc region of wild type or naturally occurring IgG and a polypeptide comprising an Fc region of IgG comprising at least one amino acid mutation compared to the wild type or naturally occurring IgG; 3) measuring the binding activity of the non-naturally occurring Fcγ receptors obtained in 1) to the polypeptide comprising the Fc region of the wild type or naturally occurring IgG; 4) measuring the binding activity of the non-naturally occurring Fcγ receptors obtained in 1) to the polypeptide comprising the Fc region of IgG comprising at least one amino acid mutation; and 5) selecting from the non-naturally occurring Fcγ receptors obtained in 1) a non-naturally occurring Fcγ receptor having essentially no binding activity to the polypeptide comprising the Fc region of wild type or naturally occurring IgG and which binds to the polypeptide comprising the Fc region of the IgG comprising at least one amino acid mutation.

In some embodiments, the Fcγ receptor is CD16A.

In some embodiments, wild type or naturally occurring Fcγ receptor is CD16A comprising the amino acid sequence shown in SEQ ID NO: 78.

In some embodiments, the Fc region of IgG comprising at least one amino acid mutation is an Fc region of human Igγ1 comprising at least one amino acid mutation compared to a wild type or naturally occurring human Igγ1 and comprises (a) a mutation from a glutamic acid to an arginine at a position corresponding to position 269 according to EU index numbering (E269R mutation) and (b) at least one mutation selected from (i) a mutation from a glutamic acid to an arginine at a position corresponding to position 294 according to EU index numbering (E294R mutation) and (ii) a mutation from a glutamic acid to a lysine at a position corresponding to position 294 according to EU index numbering (E294K mutation).

In some embodiments, the polypeptide comprising an Fc region of IgG comprising at least one amino acid mutation is an antibody. In some embodiments, the antibody is an antibody that binds to a cancer antigen.

In some embodiments, the polypeptide comprising an Fc region of IgG comprising at least one amino acid mutation is an antibody, and the method further comprises a step of contacting the antibody comprising an Fc region of IgG comprising at least one amino acid mutation obtained in 2) with an immune cell expressing the Fcγ receptor comprising at least one amino acid mutation obtained in 1) and a cell expressing an antigen to which the antibody binds, and measuring antibody-dependent cellular cytotoxicity (ADCC) activity.

In another aspect, the present invention provides a method for preparing a binding pair comprising (a) a polypeptide comprising a modified Fc region of IgG and (b) a non-naturally occurring modified Fcγ receptor. The method comprises the steps of: 1) providing a polypeptide comprising an Fc region of wild type or naturally occurring IgG and polypeptides comprising a modified Fc region of IgG, wherein the modified Fc region is non-naturally occurring and comprises at least one amino acid mutation compared to the Fc region of the wild type or naturally occurring IgG; 2) providing a wild type or naturally occurring Fcγ receptor and non-naturally occurring modified Fcγ receptors, wherein the modified Fcγ receptor comprises at least one amino acid mutation compared to the wild type or naturally occurring Fcγ receptor; 3) measuring the binding activity of each Fcγ receptor obtained in 2) to each polypeptide obtained in 1); and 4) selecting (a) a polypeptide comprising a modified Fc region that binds to the modified Fcγ receptor and has essentially no binding activity to the wild type or naturally occurring Fcγ receptor, and (b) a modified Fcγ receptor that binds to the polypeptide comprising the modified Fc region and that does not bind to the Fc region of the wild type or naturally occurring IgG.

In some embodiments, the Fcγ receptor is CD16A.

In some embodiments, wild type or naturally occurring CD16A contains the amino acid sequence shown in SEQ ID NO: 78.

In some embodiments, the polypeptide comprising the modified Fc of IgG is an antibody. In some embodiments, the antibody is an antibody that binds to a cancer antigen.

In some embodiments, the polypeptide comprising a modified Fc region of IgG selected in 4) is an antibody, and the method further comprises a step of contacting the antibody with an immune cell expressing the modified Fcγ receptor selected in 4) and a cell expressing an antigen to which the antibody binds, and measuring antibody-dependent cellular cytotoxicity (ADCC) activity.

It is expected that the present invention can provide combinations of mutagenized Fcγ receptors and mutagenized Fc regions showing specific binding patterns and an immunotherapy using these combinations in which endogenous molecules do not diminish drug efficacy.

### BRIEF DESCRIPTION OF THE DRAWINGS

FIG. 1 depicts the binding activity of wild type Fc or mutant Fc type anti-HER2 antibody to CD16V or CD16V mutants. The vertical axis represents the difference between the absorbance at 450 nm and the absorbance at reference wavelength 570 nm, and the horizontal axis represents the CD16V or CD16V mutant concentration (ng/mL).

FIG. 2 depicts the binding activity of wild type Fc or mutant Fc type anti-HER2 antibody to CD16V or CD16V mutants. The vertical axis represents the difference between the absorbance at 450 nm and the absorbance at reference wavelength 650 nm, and the horizontal axis represents the CD16V or CD16V mutant concentration (ng/mL).

FIG. 3 depicts the binding activity of wild type Fc or mutant Fc type anti-EGFR antibody to CD16V or CD16V mutants. The vertical axis represents the difference between the absorbance at 450 nm and the absorbance at reference wavelength 650 nm, and the horizontal axis represents the CD16V or CD16V mutant concentration (ng/mL).

FIG. 4 depicts the binding activity of wild type Fc or mutant Fc type anti-EpCAM antibody to CD16V or CD16V mutants. The vertical axis represents the difference between the absorbance at 450 nm and the absorbance at reference wavelength 650 nm, and the horizontal axis represents the CD16V or CD16V mutant concentration (ng/mL).

FIG. 5 depicts the binding activity of wild type Fc or mutant Fc type anti-HER2 antibody to CD16V or CD16V mutants under anti-KLH antibody competitive conditions. The vertical axis represents the difference between the absorbance at 450 nm and the absorbance at reference wavelength 650 nm, and the horizontal axis represents the CD16V or CD16V mutant concentration (ng/mL).

FIG. 6 depicts the expression level of CD16V or CD16V mutants in CD16V or CD16V mutant-expressing KHYG-1 cells in a flow cytometric analysis. The vertical axis represents the cell count and the horizontal axis represents the fluorescence intensity (CD16V expression level). Numbers in the figure indicate the proportion of cells expressing CD16V or a CD16V mutants.

FIG. 7 depicts the ADCC activity of KHYG-1 cells against HER2-positive SK-BR-3 cells in the presence of anti-HER2 antibody. The vertical axis represents cytotoxic activity (%), and the horizontal axis represents the antibody concentration (ng/mL).

FIG. 8 depicts the anti-HER2 antibody-induced ADCC activity of KHYG-1 cells against HER2-positive SK-BR-3 cells in the presence of human serum. The vertical axis represents cytotoxic activity (%).

FIG. 9 depicts the cytotoxic activity of CD16V CAR-T against HER2-positive SK-BR-3 cells in the presence of anti-HER2 antibody. The vertical axis represents cytotoxic activity (%), and the horizontal axis represents the antibody concentration (ng/mL).

### DETAILED DESCRIPTION OF THE INVENTION

The following is a detailed description of the present invention. Note that the present invention is not limited to this description. In the present specification, the scientific and technical terminology used in connection to the present invention have the meanings commonly understood by those skilled in the art unless otherwise defined.

### I. Definitions

As used herein, the term “antibody” means an immunoglobulin and refers to a biomolecule including two heavy chains (H chains) and two light chains (L chains) stabilized by a disulfide bond. A heavy chain consists of a heavy chain variable region (VH), heavy chain constant regions (CH1, CH2, CH3) and a hinge region located between CH1 and CH2, and a light chain consists of a light chain variable region (VL) and a light chain constant region (CL). Among these, a variable region fragment (Fv) composed of a VH and a VL is the region that directly participates in antigen binding and provides diversity to the antibody. Among the variable regions, regions that come into direct contact with the antigen have especially significant changes and are known as complementarity-determining regions (CDRs). A portion outside of a CDR with relatively few mutations is known as a framework region (FR). Light chain and heavy chain variable regions each have three CDRs, which are known as heavy chains CDR1 to CDR3 and light chains CDR1 to CDR3 in sequential order from the N-terminal side, respectively.

As used herein, the term, “IgG” refers to one of five classes of immunoglobulins (IgG, IgM, IgA, IgD and IgE). IgG has IgG1, IgG2, IgG3 and IgG4 subclasses, and their corresponding heavy chains are referred to as Igγ1, Igγ2, Igγ3 and Igγ4.

As used herein, the term “Fc region” refers to a region consisting of a hinge region, CH2, and CH3 in a heavy chain of the antibody, or a region consisting of CH2 and CH3 in a heavy chain of the antibody. Fc region may contain a polymorphism. (Kabat et al., Sequences of Proteins of Immunological Interest, 5th Ed., 1991, NIH Publication No. 91-3242)

As used herein, the term “antigen-binding fragment” means a fragment of an antibody capable of binding to an antigen. Specific examples of antigen-binding fragments include Fab consisting of VL, VH, CL and CH1 regions; F(ab′)2 in which two Fabs are linked by a disulfide bond in the hinge region; Fv consisting of VL and VH; scFv that is a single chain antibody in which VL and VH are linked by an artificial polypeptide linker; and bispecific antibodies such as diabodies, single-chain diabodies (scDb), tandem scFv, and leucine zippers.

As used herein, the term “human antibody” refers to an antibody having a human immunoglobulin amino acid sequence. In the present specification, a “humanized antibody” refers to an antibody in which some, most, or all of the amino acid residues other than CDRs have been replaced with amino acid residues derived from a human immunoglobulin molecule. There are no particular restrictions on the humanization method, and humanized antibodies can be prepared, for example, with reference to U.S. Pat. Nos. 5,225,539 or 6,180,370. The entire contents of each of the foregoing patents are incorporated herein by reference.

In addition to normal full-length antibodies, antibodies include antibodies of various formats such as one-armed antibodies constructed by combining a full-length antibody, antigen-binding fragment and/or Fc region (Proceedings of the National Academy of Sciences, (2013) 110 (32), pp. E2987-2996) and bispecific antibodies (Nature Reviews Drug Discovery, (2019)18, pp. 585-608).

The amino acid residue numbers for antibodies used in the present specification are pecified using EU index numbering (Kabat et al., Sequences of Proteins of Immunological Interest, 5th Ed., 1991, NIH Publication No. 91-3242) and can be defined according to this numbering system.

As used herein, the term “Fcγ receptor (FcγR)” refers to a protein belonging to an immunoglobulin superfamily that is expressed in many immune cells. FcγR is a receptor protein for the Fc region of IgG and has binding activity for the Fc region of IgG. FcγR includes FcγRI (CD64), FcγRIIA (CD32A), FcγRIIB (CD32B), FcγRIIIA (CD16A), and FcγRIIIB (CD16B). FcγRI (CD64) expressed in macrophages and dendritic cells is known to strongly bind to the Fc region of IgG, and FcγRIIA (CD32A), FcγRIIB (CD32B) and FcγRIIC (CD32C) expressed on monocytes and neutrophils as well as FcγRIIIA (CD16A) and FcγRIIIB (CD16B) expressed in macrophages and NK cells are known to weakly bind to the Fc region of IgG. CD16A is known to be involved in the initiation of ADCC (described below) by binding to the Fc region of IgG.

As used herein, the term “antibody-dependent cellular cytotoxicity (ADCC) action” refers to one of the effector actions attributable to the Fc region of an antibody. ADCC is the action by which immune cells destroy target cells through the binding of an antibody to an antigen on the target cells and to immune cells such as macrophages and NK cells. The binding of an antibody to an immune cell occurs via the Fc region of the antibody and the Fcγ receptor of the immune cell. ADCC is induced by the binding of the Fc region to Fcγ receptor IIIA (CD16A).

As used herein, the term, “combined use” refer to simultaneous or separate administration of different types of active pharmaceutical ingredients to the same subject for treatment purposes. During combined use, the different types of active pharmaceutical ingredients may be included in the same composition or included separately in different compositions.

As used herein, the term “pharmaceutical composition” refers to a single composition containing one or more pharmaceutically active ingredients. “Combination drug” means a combination of pharmaceutical compositions in which different active pharmaceutical ingredients are contained separately in different compositions.

As used herein, the term, “naturally occurring” refers to existing in nature without any artificial or man-made modifications. “Naturally occurring” and “wild type” may be used interchangeably. In the present specification, “non-naturally occurring” means an artificial or man-made product that does not exist naturally in nature, such as a product of the present invention comprising at least one amino acid mutation compared to its wild type or naturally occurring product. In the context of non-naturally occurring polypeptides and Fcγ receptors of the present invention, “non-naturally occurring” may also refer to essentially no binding to its wild type or naturally occurring counterpart but binding to a non-naturally occurring counterpart comprising at least one amino acid mutation compared to the wild type or naturally occurring counterpart.

### II. Polypeptides of the Present Invention

The present invention provides polypeptides comprising an Fc region of IgG, wherein the Fc region comprises at least one amino acid mutation, and the polypeptide has essentially no binding activity to a wild type Fcγ receptor and binds to an Fcγ receptor comprising at least one amino acid mutation.

The polypeptide comprises an Fc region of IgG. In some embodiments, the Fc region of IgG is the Fc region of human IgG, and may contain a polymorphism. In some embodiments, the Fc region of IgG is the Fc region of human Igγ1. The amino acid sequence of the Fc region can be easily obtained by those skilled in the art from a public database such as UniProt. The polypeptide of the present invention may be any form of polypeptide as long as it contains an Fc region of IgG. For example, a polypeptide of the present invention may be an antibody and an Fc fusion protein of a biomolecule or a fragment thereof and an Fc region.

In some embodiments, the polypeptide is an antibody. In some embodiments, the polypeptide is a human antibody and a humanized antibody. In the present invention, “antibody” includes ordinary IgG type antibodies as well as antibodies of various formats such as one-armed antibodies and bispecific antibodies as long as they have an Fc region.

In some embodiments, the Fc region is a modified Fc region of IgG, and the Fc region is non-naturally occurring and comprises at least one amino acid mutation compared to an Fc region of a wild type or naturally occurring IgG. In the present invention, “amino acid mutation” in the Fc region refers to a substitution, deletion, or insertion of an amino acid at a predetermined amino acid position in the Fc region. In the present invention, the polypeptide has essentially no binding activity to a wild type Fcγ receptor and binds to an Fcγ receptor comprising at least one amino acid mutation. The amino acid mutation in the Fc region of a polypeptide of the present invention may be any amino acid mutation as long as the polypeptide of the present invention exhibits these binding properties. In an embodiment of the present invention, the amino acid mutation in the Fc region is an amino acid mutation in an amino acid residue within the Fc region involved in binding of the Fc region to a wild type Fcγ receptor. In an embodiment of the present invention, the Fc region is the Fc region of human Igγ1, and the amino acid mutation in the Fc region is an amino acid mutation in at least one or more amino acid positions selected from the amino acids in amino acid positions 233, 234, 235, 236, 237, 239, 265, 269, 294, 297, 299, 328, and 329 within the human Igγ1 constant region (Nature (2000) 406, pp. 267-273, Proceedings of the National Academy of Sciences of the United States of America (2015) 112, pp. 833-838) according to EU index numbering. In an embodiment of the present invention, the Fc region is the Fc region of human Igγ1, and the amino acid mutation in the Fc region is an amino acid mutation in at least one or more amino acid positions selected from the amino acids in amino acid positions 269 and 294 within the human Igγ1 constant region according to EU index numbering. In an embodiment of the present invention, the Fc region is the Fc region of human Igγ1, and the amino acid mutation in the Fc region is an amino acid mutation in the one or two amino acid positions selected from the amino acids in amino acid positions 269 and 294 within the human Igγ1 constant region according to EU index numbering. In an embodiment of the present invention, the human Igγ1 constant region prior to introduction of a mutation contains the amino acid sequence shown in SEQ ID NO: 24. In an embodiment of the present invention, the human Igγ1 Fc region prior to introduction of a mutation contains sequence 1 to 330 in the amino acid sequence shown in SEQ ID NO: 24.

In a polypeptide of the present invention, the “amino acid mutation” in the target Fcγ receptor refers to a substitution, deletion, or insertion of an amino acid at a predetermined amino acid position in the corresponding wild type Fcγ receptor. Wild type Fcγ receptor refers to a naturally occurring Fcγ receptor that may contain a polymorphism.

An Fcγ receptor comprising at least one amino acid mutation comprises an amino acid sequence comprising at least one amino acid mutation compared to the amino acid sequence of the wild type Fcγ receptor. In an embodiment of the present invention, an Fcγ receptor comprising at least one amino acid mutation comprises an amino acid sequence having at least 90%, 95%, 96%, 97%, 98%, or 99% amino acid sequence identity with the amino acid sequence of the wild type Fcγ receptor. In an embodiment of the present invention, an Fcγ receptor comprising at least one amino acid mutation contains 5, 4, 3, 2, or 1 amino acid mutations relative to the amino acid sequence of the wild type Fcγ receptor.

As used herein, the term “identity” refers to the identity value obtained using an EMBOSS Needle (Nucleic Acids Res. (2015) 43, pp. W580-W584) by the parameters provided by default. These parameters are as follows.
Gap Open Penalty=10
Gap Extend Penalty=0.5
Matrix=EBLOSUM62

The binding properties of a polypeptide of the present invention with an Fcγ receptor can be confirmed using any known binding activity measuring method. For example, binding activity can be measured using an enzyme-linked immunosorbent assay (ELISA). In one example, the following steps can be performed to confirm the binding properties of a polypeptide of the present invention with an Fcγ receptor. A wild type Fcγ receptor and an Fcγ receptor containing at least one amino acid mutation are prepared as the Fcγ receptors to be targeted by a polypeptide of the present invention. A polypeptide (e.g., an antibody) comprising an Fc region comprising at least one amino acid mutation is prepared. The protein to be targeted by the polypeptide (e.g., antigen protein) is immobilized on an ELISA plate and the polypeptide is added and reacted with this. After reacting with the polypeptide, each Fcγ receptor is added and reacted. After these reactions, a secondary antibody such as an anti-Fcγ receptor antibody labeled with an enzyme such as horseradish peroxidase (HRP) is reacted. After these reactions, a washing operation is performed, and binding of the secondary antibody is identified by measuring activity using a reagent that detects this activity (such as a TMB reagent (DAKO, Cat. S1599) in the case of HRP labeling) so that it can be evaluated whether or not the antibody including an Fc region containing at least one amino acid mutation binds to the wild type Fcγ receptor and to the Fcγ receptor containing the amino acid mutation or mutations. The specific method of evaluation used can be the method described in Example 4 below.

As used herein, the phrase “having essentially no binding activity” to a wild type Fcγ receptor refers to the binding activity of a polypeptide of the present invention to a wild type Fcγ receptor is not significantly higher than the binding activity of the polypeptide of the present invention to the Fcγ receptor containing at least one amino acid mutation to which the polypeptide of the present invention binds. In an embodiment of the present invention, when “having essentially no binding activity” to a wild type Fcγ receptor is measured using the ELISA method, this is 10% or less, 5% or less, 3% or less, 2% or less, 1% or less, 0.5% or less, 0.1% or less, or 0.05% or less of the binding activity to the Fcγ receptor containing at least one amino acid mutation used as the control.

The Fcγ receptor targeted by a polypeptide of the present invention can be selected by those skilled in the art based on the intended use for the polypeptide of the present invention and other factors. In an embodiment of the present invention, the Fcγ receptor can be FcγRI (CD64), FcγRIIA (CD32A), FcγRIIB (CD32B), FcγRIIIA (CD16A), or FcγRIIIB (CD16B). In an embodiment of the present invention, the Fcγ receptor targeted by a polypeptide of the present invention is CD16. In an embodiment of the present invention, the Fcγ receptor is CD16A. Wild type CD16A includes, but not limited to, two polymorphisms: CD16A V158 and CD16A F158. In an embodiment of the present invention, the wild type CD16A is CD16A V158.

In an embodiment of a polypeptide of the present invention, the target Fcγ receptor is CD16A and the wild type CD16A contains the amino acid sequence shown in SEQ ID NO: 78.

In an embodiment of a polypeptide of the present invention, the target Fcγ receptor is CD16A comprising at least one amino acid mutation, and the CD16A comprising at least one amino acid mutation comprises at least one mutation selected from a mutation from the lysine to the aspartic acid at a position corresponding to position 131 in SEQ ID NO: 78 (K131D mutation), a mutation from the lysine to the glutamic acid at a position corresponding to position 128 in SEQ ID NO: 78 (K128E mutation), and a mutation from the lysine to the glutamic acid at a position corresponding to position 131 in SEQ ID NO: 78 (K131E mutation). In an embodiment of the present invention, the CD16A comprising at least one amino acid mutation contains one or both of the K131D mutation and the K128E mutation. In an embodiment of the present invention, the CD16A comprising at least one amino acid mutation comprises one or both of the K131E mutation and the K128E mutation. In an embodiment of the present invention, the CD16A comprising at least one amino acid mutation comprises the K131D mutation and at least one mutation selected from a mutation from the asparagine to the glutamine at a position corresponding to position 38 in SEQ ID NO: 78 (N38Q mutation) and a mutation from the asparagine to the glutamine at a position corresponding to position 74 in SEQ ID NO: 78 (N74Q mutation).

When used in the present specification, the term “position corresponding” to a position in SEQ ID NO: 78 refers to the amino acid position in a CD16A is aligned with the same position as the amino acid position in SEQ ID NO: 78 when the amino acid sequence in the CD16A is aligned with the amino acid sequence in SEQ ID NO: 78 using a sequence alignment program such as BLAST.

In an embodiment of the present invention, the CD16A comprising at least one amino acid mutation comprises the amino acid sequence shown in SEQ ID NO: 80, SEQ ID NO: 82, SEQ ID NO: 84, SEQ ID NO: 86, or SEQ ID NO: 88.

In an embodiment of the present invention, the polypeptide of the present invention has essentially no binding activity to CD16A comprising the amino acid sequence of SEQ ID NO: 78 and binds to CD16A comprising the amino acid sequence shown in SEQ ID NO: 80, SEQ ID NO: 82, SEQ ID NO: 84, SEQ ID NO: 86, or SEQ ID NO: 88.

In an embodiment of the present invention, the polypeptide comprises the Fc region of human Igγ1, and the Fc region comprises an amino acid mutation in at least one or more amino acid positions selected from the amino acids in amino acid positions 269 and 294 within the human Igγ1 constant region according to EU index numbering. In an embodiment of the present invention, the polypeptide comprises the Fc region of human Igγ1, and the Fc region comprises an amino acid mutation in the one or two amino acid positions selected from the amino acids in amino acid positions 269 and 294 within the human Igγ1 constant region according to EU index numbering.

In an embodiment of the present invention, the polypeptide comprises an Fc region of human Igγ1, and the Fc region comprises a mutation from the glutamic acid to the arginine at a position corresponding to position 269 according to EU index numbering (E269R mutation) and at least one mutation selected from a mutation from the glutamic acid to the arginine at a position corresponding to position 294 according to EU index numbering (E294R mutation) and a mutation from the glutamic acid to the lysine at a position corresponding to position 294 according to EU index numbering (E294K mutation).

Amino acid sequence information on a polypeptide of the present invention and the Fcγ receptor to be targeted by the polypeptide can be obtained from a public database such as UniProt, and they can be easily prepared based on this amino acid sequence information using any method known to those skilled in the art or using the method described in the present specification.

When a polypeptide of the present invention is an antibody, an antibody against any antigen can be used. When a polypeptide of the present invention is an antibody, the antibody may be obtained by immunization with the target antigen according to any antibody preparation method known to those skilled in the art, or may be prepared by introducing an amino acid mutation into the Fc region of a known antibody.

In an embodiment of the present invention, the polypeptide is an antibody that binds to a cancer antigen. Known antibodies that can be used to prepare a polypeptide of the present invention include an anti-CD19 antibody (tafacitamab, Drug Bank Accession Number: DB15044), an anti-HER2 antibody, an anti-EpCAM antibody, or an anti-EGFR antibody.

In an embodiment of the present invention, the polypeptide is an anti-CD19 antibody, an anti-HER2 antibody, an anti-EpCAM antibody, or an anti-EGFR antibody comprising an Fc region of human Igγ1, and the Fc region comprises a mutation from the glutamic acid to the arginine at position 269 in the human Igγ constant region according to EU index numbering (E269R mutation) and at least one mutation selected from a mutation from the glutamic acid to the arginine at position 294 (E294R mutation) and a mutation from the glutamic acid to the lysine in the human Igγ constant region at position 294 according to EU index numbering (E294K mutation).

### III. Cells Expressing CD16A Comprising Amino Acid Mutations

The present invention also provides a CD16A-expressing cell that can be used in combination with a polypeptide of the present invention.

In one aspect, the present invention provides a cell expressing a non-naturally occurring CD16A comprising at least one amino acid mutation compared to a wild type or naturally occurring CD16A, wherein the at least one amino acid mutation is selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K131E mutation), and wherein the non-naturally occurring CD16A comprises an amino acid sequence having 90% or more amino acid sequence identity with SEQ ID NO: 78.

In an embodiment of the present invention, the CD16A expressed by a CD16A-expressing cell of the present invention comprises one or both of the K131D mutation and the K128E mutation. In an embodiment of the present invention, the CD16A expressed by a CD16A-expressing cell of the present invention comprises one or both of the K131E mutation and the K128E mutation. In an embodiment of the present invention, the CD16A expressed by a CD16A-expressing cell of the present invention comprises the K131D mutation and at least one mutation selected from a mutation from the asparagine to the glutamine at a position corresponding to position 38 in SEQ ID NO: 78 (N38Q mutation) and a mutation from the asparagine to the glutamine at a position corresponding to position 74 in SEQ ID NO: 78 (N74Q mutation).

In an embodiment of the present invention, the CD16A expressed by a CD16A-expressing cell of the present invention comprises an amino acid sequence having at least 95%, 96%, 97%, 98%, or 99% amino acid sequence identity with the amino acid sequence of SEQ ID NO: 78.

In an embodiment of the present invention, the CD16A expressed by a CD16A-expressing cell of the present invention comprises the amino acid sequence shown in SEQ ID NO: 80, SEQ ID NO: 82, SEQ ID NO: 84, SEQ ID NO: 86, or SEQ ID NO: 88.

The cell used as a CD16A-expressing cell of the present invention can be any cell as long as the immune response can be regulated (for example, induced or suppressed) by use with a polypeptide of the present invention. The cell may be an immune cell of the innate immune system or acquired immune system, and examples include NK cells, NKT-cells, macrophages, microglia, osteoclasts, granulocytes (including neutrophils, eosinophils, and basophils), monocytes, dendritic cells, T cells, and B cells, and the like. In an embodiment of the present invention, the cell is a cell of human origin. In an embodiment of the present invention, the cell is a human immune cell. In an embodiment of the present invention, the cell is a human NK cell or human T cell.

In an embodiment of the present invention, the cell used as the CD16A-expressing cell of the present invention may be a stem cell, including, but not limited to a pluripotent stem cell, hematopoietic stem cell, adult stem cell, fetal stem cell, mesenchymal stem cell, postpartum stem cell, multipotent stem cell, or embryonic germ cell, or a cell derived from such stem cell, such as an immune cell described above. In an embodiment of the invention, the stem cell is a pluripotent stem cell. A pluripotent stem cell may be an induced pluripotent stem cell (iPS cell) or embryonic stem cell (ES cell), and may be of human origin. The iPS cell or ES cell can be prepared by those skilled in the art using any known method. The method used to differentiate a stem cell such as an iPS cell or ES cell into a CD16A-expressing cell of the present invention can be any method known to those skilled in the art. In an embodiment of the present invention, the CD16A-expressing cell of the present invention is a human immune cell derived from a pluripotent stem cell. In an embodiment of the present invention, the CD16A-expressing cell is a human NK cell or human T cell derived from a pluripotent stem cell.

In an embodiment of the present invention, the stem cell may be a universal donor cell in which the stem cell has been gene-edited to escape allogeneic responses and lysis by NK cells. The universal donor cell and a cell derived from the universal donor cell may comprise a genetically engineered disruption in a beta-2 microglobulin (B2M) gene to eliminate expression of HLA class I molecules as described, for example, in WO 2012/145384, which is herein incorporated by reference in its entirety. The universal donor cell and a cell derived from the universal donor cell may further comprise a polynucleotide capable of encoding a single chain fusion human leukocyte antigen (HLA) class I protein comprising at least a portion of the B2M protein covalently linked, either directly or via a linker sequence, to at least a portion of an HLA-1α chain. In an embodiment, the HLA-1α chain is selected from HLA-A, HLA-B, HLA-C, HLA-E, HLA-F, and HLA-G. See also WO 2012/145384. The universal donor cell and a cell derived from the universal donor cell may additionally contain a genetically engineered disruption in a HLA class II-related gene by knocking out one or more of the transcription factors required for the expression of the HLA class II gene, such as regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB, as described, for example, in WO 2013/158292, which is also herein incorporated by reference in its entirety. The cell may further comprise one or more polynucleotides encoding a single chain fusion HLA class II protein or an HLA class II protein. See also WO 2013/158292. In an embodiment of the invention, the universal donor cell and a cell derived from the universal donor cell express a CD16A comprising at least one amino acid mutation. In an embodiment of the present invention, the universal donor cell is an iPS cell or ES cell, and the CD16A-expressing cell is a cell derived from such universal donor cell, such as an immune cell described above. In an embodiment of the present invention, the CD16A-expressing cell is a human immune cell derived from a universal donor cell. In an embodiment of the present invention, the CD16A-expressing cell is a human NK cell or human T cell derived from a universal donor cell.

In an embodiment of the present invention, the CD16A-expressing cell can be isolated and/or purified. In an embodiment of the present invention, the CD16A-expressing cell is an immortalized cell or established cell line, and this immortalized cell or established cell line can be prepared by those skilled in the art using any known method. In an embodiment of the present invention, the cell used as the CD16A-expressing cell of the present invention is an immortalized cell or established cell line of human origin. In an embodiment of the present invention, the cell used as the CD16A-expressing cell of the present invention is a cell derived from the patient.

In an embodiment of the present invention, the CD16A-expressing cell of the present invention is a cell that exogenously expresses the CD16A. The CD16A-expressing cell of the present invention can be prepared by introducing a gene encoding CD16A containing a desired amino acid mutation into the cell. For example, the gene can be synthesized using the phosphoramidite method based on the nucleotide sequence or can be prepared by combining DNA fragments obtained from a cDNA library using polymerase chain reaction (PCR). The target gene can be introduced into the cell using an expression vector containing the gene in the form of cDNA. The gene may also be introduced into the cell with a polynucleotide in the form of mRNA. Alternatively, the target gene may be directly introduced into the cell using a method such as electroporation or lipofection. The cell may be cultured and proliferated after introduction of the target gene into the cell.

There are no particular restrictions on the expression vector used to prepare a CD16A-expressing cell of the present invention as long as it can express a target protein such as CD16A containing an amino acid mutation in the target cell. Examples of expression vectors that may be used include a plasmid vector (such as the pcDNA series from Thermo Fisher Scientific, the pALTER®-MAX Vector from Promega, and the pHEK293 Ultra Expression Vector from Takara) or a viral vector (such as a lentivirus, adenovirus, retrovirus, or adeno-associated virus). In the production of a viral vector, a pLVSIN-CMV/EF1α vector (Takara Bio) or pLenti vector (Thermo Fisher Scientific) used in the production of a lentivirus can be employed.

The expression vector can include a start codon and a stop codon. In this case, it may include an enhancer sequence, a non-translated region, a splicing junction, a polyadenylation site, or a replicable unit. The expression vector may include a gene that can serve as a marker for confirming expression of the target gene (such as a drug resistant gene, a gene encoding a reporter enzyme, or a gene encoding a fluorescent protein).

Culturing can be performed by a known method in order to obtain or maintain CD16A-expressing cells of the present invention. Examples of basal media that can be used include a MEM medium (Science (1955) 122, pp.501-504), a DMEM medium (Virology (1959) 8, pp. 396-397), a RPMI1640 medium (The Journal of the American Medical Association (1967) 199, pp. 519-524), a 199 medium (Proceedings of the Society for Experimental Biology and Medicine (1950) 73, pp. 1-8), a FreeStyle™293 Expression Medium (Thermo Fisher Scientific, Cat. 12338026), a CD293 Medium (Thermo Fisher Scientific, Cat. 11913019), or an Expi293™ Expression Medium (Thermo Fisher Scientific, Cat. A1435101). The culture medium may also contain serum (such as fetal bovine serum; FBS), a serum substitute (such as Knock Out Serum Replacement: KSR), fatty acids or lipids, amino acids, vitamins, growth factor, cytokines, antioxidants, 2-mercaptoethanol, pyruvic acid, a buffer, inorganic salts, and antibiotics. In an embodiment of the present invention, the medium is a serum-free medium or a chemically defined medium. The culture conditions (such as the culture time, temperature, medium pH, and CO₂ concentration) can be selected as appropriate by those skilled in the art. The pH of the medium is preferably from about 6 to 8. There are no particular restrictions on the culture temperature, but a culture of about 30 to 40° C., preferably about 37° C., can be used. The CO₂ concentration can be about 1 to 10%, and preferably about 5%. There are no particular restrictions on the culture time, but can be about 15 to 336 hours. The culture can be aerated or agitated as necessary. When an inducible promoter that is induced by a drug such as tetracycline or doxycycline is used, a step may be included to culture the cells in a medium containing the drug and then induce expression of the gene operably linked to the inducible promoter such as a cancer antigen. This step can be performed in accordance with a gene induction method using a general gene induction system.

### IV. Method For Treating and Preventing a Disease or Disorder in a Patient Using Immunotherapy

The present invention also provides a method for treating or preventing a disease or disorder in a patient using immunotherapy. The method comprises administering to a patient a polypeptide of the present invention and a cell expressing the non-naturally occurring Fcγ receptor comprising at least one amino acid mutation, wherein the polypeptide is capable of binding to said non-naturally occurring Fcγ receptor comprising at least one amino acid mutation.

In the present specification, the term “immunotherapy” refers to a method for preventing or treating autoimmune disease, cancers and infectious diseases caused by various bacteria or viruses using the functions of self/non-self-recognizing immune cells to eliminate foreign substances such as exogenous bacteria, viruses and cancer cells.

The cells used in the treatment method of the present invention are cells expressing an Fcγ receptor comprising at least one amino acid mutation, wherein the Fcγ receptor is capable of binding to a polypeptide of the present invention. The cell expressing the Fcγ receptor can be any cell as long as the immune response can be regulated (for example, induced or suppressed) by use with a polypeptide of the present invention. The cell may be an immune cell of the innate immune system or acquired immune system, and examples include NK cells, NKT-cells, macrophages, microglia, osteoclasts, granulocytes (including neutrophils, eosinophils, and basophils), monocytes, dendritic cells, T cells, and B cells, and the like. In an embodiment of the present invention, the cell is a cell of human origin. In an embodiment of the present invention, the cell is a human immune cell. In an embodiment of the present invention, the cell is a human NK cell or human T cell.

In an embodiment of the present invention, the cell used in the treatment method of the present invention, such as an immune cell described above, may be derived from a stem cell, including, but not limited to a pluripotent stem cell, hematopoietic stem cell, adult stem cell, fetal stem cell, mesenchymal stem cell, postpartum stem cell, multipotent stem cell, or embryonic germ cell. In an embodiment of the invention, the stem cell is engineered to express an Fcγ receptor containing at least one amino acid mutation, wherein the Fcγ receptor is capable of binding to a polypeptide of the present invention. In an embodiment of the invention, the stem cell is a pluripotent stem cell. A pluripotent stem cell may be an induced pluripotent stem cell (iPS cell) or embryonic stem cell (ES cell), and may be of human origin. The iPS cell or ES cell can be prepared by those skilled in the art using any known method. The method used to differentiate a stem cell such as an iPS cell or ES cell into a cell used in the treatment method of the present invention can be any method known to those skilled in the art. In an embodiment of the present invention, the cell is a human immune cell derived from a pluripotent stem cell. In an embodiment of the present invention, the cell is a human NK cell or human T cell derived from a pluripotent stem cell.

In an embodiment of the present invention, the cell used in the treatment method of the present invention may be derived from a universal donor cell in which the stem cell has been gene-edited to escape allogeneic responses and lysis by NK cells. The universal donor cell and a cell derived from the universal donor cell may comprise a genetically engineered disruption in a beta-2 microglobulin (B2M) gene to eliminate expression of HLA class I molecules as described, for example, in WO 2012/145384, which is herein incorporated by reference in its entirety. The universal donor cell and a cell derived from the universal donor cell may further comprise a polynucleotide capable of encoding a single chain fusion human leukocyte antigen (HLA) class I protein comprising at least a portion of the B2M protein covalently linked, either directly or via a linker sequence, to at least a portion of an HLA-1α chain. In an embodiment, the HLA-1α chain is selected from HLA-A, HLA-B, HLA-C, HLA-E, HLA-F, and HLA-G. See also WO 2012/145384. The universal donor cell and a cell derived from the universal donor cell may additionally contain a genetically engineered disruption in a HLA class II-related gene by knocking out one or more of the transcription factors required for the expression of the HLA class II gene, such as regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB, as described, for example, in WO 2013/158292, which is also herein incorporated by reference in its entirety. The cell may further comprise one or more polynucleotides encoding a single chain fusion HLA class II protein or an HLA class II protein. See also WO 2013/158292. In another embodiment, the universal donor cell and a cell derived from the universal donor cell are engineered to express an Fcγ receptor containing at least one amino acid mutation, wherein the Fcγ receptor is capable of binding to a polypeptide of the present invention. In an embodiment of the present invention, the universal donor cell is an iPS cell or ES cell. In an embodiment of the present invention, the cell used in the treatment method of the present invention is a human immune cell derived from a universal donor cell. In an embodiment of the present invention, the cell used in the treatment method of the present invention is a human NK cell or human T cell derived from a universal donor cell.

In an embodiment of the present invention, the cell used in the treatment method of the present invention can be isolated and/or purified. Here, “isolation” means separation from living tissue, and “purification” means separation of the cell from one or more additional components in the tissue from which the cell was derived. In an embodiment of the present invention, the cell used in the treatment method of the present invention is an immortalized cell or established cell line, and this immortalized cell or established cell line can be prepared by those skilled in the art using any known method. In an embodiment of the present invention, the cell used in the treatment method of the present invention is an immortalized cell or established cell line of human origin. In an embodiment of the present invention, the cell used in the treatment method of the present invention is a cell derived from the patient.

The Fcγ receptor expressed in the cell used in the treatment method of the present invention is the Fcγ receptor targeted by a polypeptide of the present invention and can be selected by those skilled in the art based on the intended use for the polypeptide of the present invention and other factors. In an embodiment of the present invention, the Fcγ receptor can be FcγRI (CD64), FcγRIIA (CD32A), FcγRIIB (CD32B), FcγRIIIA (CD16A), or FcγRIIIB (CD16B). In an embodiment of the present invention, the Fcγ receptor targeted by a polypeptide of the present invention is CD16. In an embodiment of the present invention, the Fcγ receptor is CD16A. CD16A includes, but not limited to, two polymorphisms: CD16A V158 and CD16A F158. In an embodiment of the present invention, the CD16A is CD16A V158.

In an embodiment the present invention, the Fcγ receptor expressed by the cell used in the treatment method of the present invention is CD16A comprising at least one amino acid mutation selected from a mutation from the lysine to the aspartic acid at a position corresponding to position 131 in SEQ ID NO: 78 (K131D mutation), a mutation from the lysine to the glutamic acid at a position corresponding to position 128 in SEQ ID NO: 78 (K128E mutation), and a mutation from the lysine to the glutamic acid at a position corresponding to position 131 in SEQ ID NO: 78 (K13 1E mutation). In an embodiment of the present invention, the Fcγ receptor expressed by the cell used in the treatment method of the present invention is CD16A comprising one or both of the K131D mutation and the K128E mutation. In an embodiment of the present invention, the Fcγ receptor expressed by the cell used in the treatment method of the present invention is CD16A comprising one or both of the K13 1E mutation and the K128E mutation. In an embodiment of the present invention, the Fcγ receptor expressed by the cell used in the treatment method of the present invention is CD16A comprising the K131D mutation and at least one mutation selected from a mutation from the asparagine to the glutamine at a position corresponding to position 38 in SEQ ID NO: 78 (N38Q mutation) and a mutation from the asparagine to the glutamine at a position corresponding to position 74 in SEQ ID NO: 78 (N74Q mutation).

In an embodiment of the present invention, the Fcγ receptor expressed by the cell used in the treatment method of the present invention is CD16A comprising the amino acid sequence shown in SEQ ID NO: 80, SEQ ID NO: 82, SEQ ID NO: 84, SEQ ID NO: 86, or SEQ ID NO: 88.

In an embodiment of the present invention, the treatment method comprises administering to a patient: (A) a polypeptide comprising an Fc region of human Igγ1, wherein the Fc region comprises a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation); and (B) a cell selected from: (i) a cell expressing a CD16A containing at least one mutation selected from (a) a mutation from the lysine to the aspartic acid at a position corresponding to position 131 in SEQ ID NO: 78 (K131D mutation), (b) a mutation from the lysine to the glutamic acid at a position corresponding to position 128 in SEQ ID NO: 78 (K128E mutation), and (c) a mutation from the lysine to the glutamic acid at a position corresponding to position 131 in SEQ ID NO: 78 (K131E mutation); (ii) a cell expressing a CD16A containing one or both of the K131D mutation and the K128E mutation; (iii) a cell expressing a CD16A containing one or both of the K131E mutation and the K128E mutation; and (iv) a cell expressing a CD16A containing the K131D mutation and at least one mutation selected from a mutation from the asparagine to the glutamine at a position corresponding to position 38 in SEQ ID NO: 78 (N38Q mutation) and a mutation from the asparagine to the glutamine at a position corresponding to position 74 in SEQ ID NO: 78 (N74Q mutation).

In an embodiment of the present invention, the treatment method comprises administering to a patient: (A) a polypeptide comprising an Fc region of human Igγ1, wherein the Fc region comprises a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation); and (B) a cell expressing a CD16A comprising the amino acid sequence shown in SEQ ID NO: 80, SEQ ID NO: 82, SEQ ID NO: 84, SEQ ID NO: 86, or SEQ ID NO: 88.

In an embodiment of the present invention, the polypeptide used in the treatment method is an antibody. In an embodiment of the present invention, the antibody is an antibody that binds to a cancer antigen. In an embodiment of the present invention, the polypeptide used in the treatment method is an anti-CD19 antibody, anti-HER2 antibody, anti-EpCAM antibody, or anti-EGFR antibody having an Fc region that is the Fc region of human Igγ1 and comprising a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation).

The cell expressing the Fcγ receptor comprising at least one amino acid mutation used in the treatment method of the present invention can be prepared by introducing a gene encoding the Fcγ receptor comprising the target amino acid mutation into the cell. The gene encoding the Fcγ receptor comprising the amino acid mutation can be prepared using a standard molecular biology and/or chemical technique in which the nucleotide sequence encoding the amino acid sequence of the target Fcγ receptor is acquired from the NCBI Ref Seq ID or GenBank Accession number, and the sequence of the gene encoding the Fcγ receptor comprising the target amino acid mutation is designed using this nucleotide sequence. The introduction of the gene into the cell and culturing of the cell can be conducted using any method known to those skilled in the art, and the methods described herein.

There are no particular restrictions on the diseases targeted by the treatment method of the present invention, which can be bacterial infections, viral infections, autoimmune disease, and cancers. In an embodiment of the present invention, the treatment method is used to treat or prevent cancer. In an embodiment of the present invention, the treatment method is a treatment or prevention method for patients using cancer immunotherapy. Here, “cancer immunotherapy” refers to a method for preventing or treating cancer by activating or proliferating immune cells that play a role in identifying cancer cells occurring in the body as foreign substances by immunity and eliminating them.

In the method of the present invention, a polypeptide of the present invention and cells expressing an Fcγ receptor comprising at least one amino acid mutation to which the polypeptide binds can be administered to a subject in need of immunotherapy using any method known to those of skill in the art. When a polypeptide of the present invention is administered to a subject, it can be administered to the subject in the form of a pharmaceutical composition comprising the polypeptide of the present invention and a pharmaceutically acceptable excipient. When cells expressing an Fcγ receptor comprising at least one amino acid mutation to which the polypeptide binds are administered to a subject, they can be administered to the subject in the form of a pharmaceutical composition comprising the cells and a pharmaceutically acceptable excipient. In a treatment method of the present invention, a pharmaceutical composition comprising a polypeptide of the present invention, cells expressing an Fcγ receptor comprising at least one amino acid mutation to which the polypeptide binds, and a pharmaceutically acceptable excipient can be administered to the subject. The pharmaceutical compositions as described herein can be used.

The dose and number of doses of a polypeptide of the present invention and cells expressing an Fcγ receptor comprising at least one amino acid mutation to which the polypeptide binds administered to a subject can be adjusted as appropriate depending on the target disease, the age, weight and condition of the subject being treated, the dosage form, the type and titer of the polypeptide of the present invention, and the type of cells used in the treatment method of the present invention. The dose of the polypeptide of the present invention can be, for example, about 0.001 mg/kg to 100 mg/kg. The dose of cells used in the method can be, for example, 1×10³ cells/kg to 1×10⁹ cells/kg per administration to the subject.

A polypeptide of the present invention and the cells used in the method can be administered to a subject by any appropriate route of administration, for example, by intravenous injection, intratumoral injection, intradermal injection, subcutaneous injection, intramuscular injection, intraperitoneal injection, or intraarterial injection.

In the method of the present invention, a polypeptide of the present invention and cells expressing an Fcγ receptor comprising at least one amino acid mutation to which the polypeptide binds administered to a subject can be administered simultaneously, continuously, or sequentially. In an embodiment of the present invention, administration of a polypeptide of the present invention to the subject is started and then administration of the cells used in the treatment method of the present invention is started. In an embodiment of the present invention, administration of the cells used in the treatment method of the present invention to the subject is started and then administration of a polypeptide of the present invention is started. In an embodiment of the present invention, administration of a polypeptide of the present invention to the subject is completed and then administration of the cells used in the treatment method of the present invention is started. In an embodiment of the present invention, administration of the cells used in the treatment method of the present invention to the subject is completed and then administration of a polypeptide of the present invention is started.

### V. Pharmaceutical Compositions and Combination Drugs of the Present Invention

The present invention further provides the following pharmaceutical compositions and combination drugs:
(1) a pharmaceutical composition comprising a polypeptide of the present invention and a pharmaceutically acceptable excipient;
(2) a pharmaceutical composition comprising CD16A-expressing cells of the present invention and a pharmaceutically acceptable excipient;
(3) a pharmaceutical composition comprising a polypeptide of the present invention, cells expressing an Fcγ receptor comprising at least one amino acid mutation to which the polypeptide binds, and a pharmaceutically acceptable excipient; and
(4) a combination drug of a pharmaceutical composition comprising a polypeptide of the present invention and a pharmaceutically acceptable excipient, and a pharmaceutical composition comprising cells expressing an Fcγ receptor comprising at least one amino acid mutation to which the polypeptide binds and a pharmaceutically acceptable excipient.

A combination drug of the present invention may be in the form of a kit in which each of the constituent pharmaceutical compositions is included in a single package.

A pharmaceutical composition of the present invention can be prepared by a method common in the art using an excipient common in the art, that is, a pharmaceutically acceptable excipient, or a pharmaceutically acceptable carrier. The dosage form of these pharmaceutical compositions can be a parenteral agent such as an injection or infusion. During formulation, excipients, carriers and additives appropriate to the dosage form can be used within a pharmaceutically acceptable range.

In an embodiment of the pharmaceutical composition in (1), the pharmaceutical composition in (1) is a pharmaceutical composition for combined use with cells in a treatment or prevention method for patients by immunotherapy, wherein the cells are cells expressing an Fcγ receptor containing at least one amino acid mutation to which the polypeptide binds. In an embodiment of the present invention, the cells are human immune cells. In an embodiment of the present invention, the human immune cells are human T cells or human NK cells. In an embodiment of the present invention, the cells are cells expressing CD16A containing at least one amino acid mutation. In an embodiment of the present invention, the cells are CD16A-expressing cells of the present invention.

In an embodiment of the present invention, the pharmaceutical composition in (1) can be a pharmaceutical composition comprising a polypeptide comprising an Fc region of human Igγ1, wherein the Fc region comprises a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation), and wherein the pharmaceutical composition is a pharmaceutical composition for combined use with a cell selected from:
(i) cells expressing CD16A comprising at least one mutation selected from a mutation from the lysine to the aspartic acid at a position corresponding to position 131 in SEQ ID NO: 78 (K131D mutation), a mutation from the lysine to the glutamic acid at a position corresponding to position 128 in SEQ ID NO: 78 (K128E mutation), and a mutation from the lysine to the glutamic acid at a position corresponding to position 131 in SEQ ID NO: 78 (K131E mutation); (ii) cells expressing CD16A comprising one or both of the K131D mutation and the K128E mutation; (iii) cells expressing CD16A comprising one or both of the K131E mutation and the K128E mutation; or (iv) cells expressing CD16A comprising the K131D mutation and at least one mutation selected from a mutation from the asparagine to the glutamine at a position corresponding to position 38 in SEQ ID NO: 78 (N38Q mutation) and a mutation from the asparagine to the glutamine at a position corresponding to position 74 in SEQ ID NO: 78 (N74Q mutation).

In an embodiment of the present invention, the pharmaceutical composition in (1) is a pharmaceutical composition comprising a polypeptide comprising an Fc region of human Igγ1, wherein the Fc region comprises a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation), and wherein the pharmaceutical composition is a pharmaceutical composition for combined use with cells expressing CD16A comprising the amino acid sequence shown in SEQ ID NO: 80, SEQ ID NO: 82, SEQ ID NO: 84, SEQ ID NO: 86, or SEQ ID NO: 88.

In an embodiment of the present invention, the polypeptide used in the pharmaceutical composition in (1) is an antibody. In an embodiment of the present invention, the antibody is an antibody that binds to a cancer antigen. In an embodiment of the present invention, the polypeptide used in the pharmaceutical composition in (1) is an anti-CD19 antibody, anti-HER2 antibody, anti-EpCAM antibody, or anti-EGFR antibody having an Fc region that is the Fc region of human Igγ1 and comprising a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation).

In an embodiment of the pharmaceutical composition in (2), the pharmaceutical composition in (2) is a pharmaceutical composition for combined use with a polypeptide in a treatment or prevention of a disease or disorder in a patient by immunotherapy, wherein the peptide is a polypeptide comprising an Fc region of IgG, wherein the Fc region comprises at least one amino acid mutation, and the polypeptide has essentially no binding activity to wild type CD16A and binds to CD16A comprising at least one amino acid mutation expressed in the cells. In an embodiment of the present invention, the polypeptide comprises an Fc region of human Igγ1, and the Fc region contains a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation). In an embodiment of the present invention, the polypeptide is an antibody. In an embodiment of the present invention, the antibody is an antibody that binds to a cancer antigen. In an embodiment of the present invention, the polypeptide is an anti-CD19 antibody, anti-HER2 antibody, anti-EpCAM antibody, or anti-EGFR antibody having an Fc region that is the Fc region of human Igγ1 and comprising a mutation from the glutamic acid to the arginine at position 269 according to EU index numbering (E269R mutation) and at least one mutation selected from a mutation from the glutamic acid to the arginine at position 294 in the human Igγ1 constant region according to EU index numbering (E294R mutation) and a mutation from the glutamic acid to the lysine at position 294 in the human Igγ1 constant region according to EU index numbering (E294K mutation).

In an embodiment of the pharmaceutical composition in (3), the pharmaceutical composition in (3) is a pharmaceutical composition for prevention and treatment of a disease or disorder in a patient using immunotherapy. In an embodiment of the combined drug in (4), the combined drug in (4) is a combined drug for prevention and treatment of a disease or disorder in a patient using immunotherapy.

In an embodiment of the pharmaceutical composition in (3) and the combined drug in (4), the cell expressing an Fcγ receptor comprising at least one amino acid mutation to which a polypeptide of the present invention binds is a human immune cell. In an embodiment of the present invention, the human immune cell is a human T cell or a human NK cell. In an embodiment of the present invention, the cell is a cell expressing CD16A comprising at least one amino acid mutation. In an embodiment of the present invention, the cell is a CD16A-expressing cell of the present invention.

In an embodiment of the pharmaceutical composition in (3) and the combined drug in (4), when the cell expressing an Fcγ receptor comprising at least one amino acid mutation to which a polypeptide of the present invention binds is a CD16A-expressing cell of the present invention, the polypeptide of the present invention comprises an Fc region of IgG, and the Fc region comprises a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region according to EU index numbering (E294R mutation), and a mutation from the glutamic acid to the lysine at position 294 in the human Igγ1 constant region according to EU index numbering (E294K mutation). In an embodiment of the present invention, the polypeptide is an antibody. In an embodiment of the present invention, the antibody is an antibody that binds to a cancer antigen. In an embodiment of the present invention, the polypeptide is an anti-CD19 antibody, anti-HER2 antibody, anti-EpCAM antibody, or anti-EGFR antibody having an Fc region that is the Fc region of human Igγ1 and containing a mutation from the glutamic acid to the arginine at position 269 in the human Igγ1 constant region according to EU index numbering (E269R mutation) and at least one mutation selected from a mutation from the glutamic acid to the arginine at position 294 in the human Igγ1 constant region in the human Igγ1 constant region (E294R mutation) and a mutation from the glutamic acid to the lysine at position 294 in the human Igγ1 constant region according to EU index numbering (E294K mutation).

While there are no particular restrictions, the pharmaceutical composition or combined drug of the present invention can be used to treat or prevent bacterial infections, viral infections, autoimmune disease, and cancers.

The present invention also provides the following combinations of CD16A-expressing cells and polypeptide as described herein.

(1) A polypeptide of the present invention for treatment or prevention of a disease or disorder in a patient using immunotherapy in combined use with a cell expressing an Fcγ receptor comprising at least one amino acid mutation to which the polypeptide of the present invention binds. In an embodiment of the present invention, the polypeptide is used to treat or prevent cancer.

(2) A CD16A-expressing cell of the present invention for treatment or prevention of a disease or disorder in a patient using immunotherapy in combined use with a polypeptide comprising the Fc region of IgG having essentially no binding activity to wild type CD16A but is capable of binding to the CD16A comprising at least one amino acid mutation expressed in a CD16A-expressing cell of the present invention. In an embodiment of the present invention, the CD16A-expressing cell is used to treat or prevent cancer.

(3) A use of polypeptide of the present invention in the production of a pharmaceutical composition for treatment or prevention of a disease or disorder in a patient using immunotherapy in combined use with a cell expressing an Fcγ receptor comprising at least one amino acid mutation to which the polypeptide of the present invention binds. In an embodiment of the present invention, the polypeptide is used to treat or prevent cancer.

(4) A use of CD16A-expressing cell of the present invention in the production of a pharmaceutical composition for treatment or prevention of a disorder in a patient using immunotherapy in combined use with a polypeptide comprising the Fc region of IgG having essentially no binding activity to wild type CD16A but is capable of binding to the CD16A comprising at least one amino acid mutation expressed in a CD16A-expressing cell of the present invention. In an embodiment of the present invention, the CD16A-expressing cell is used to treat or prevent bacterial infections, viral infections, autoimmune disease, and/or cancer.

The embodiments related to polypeptides of the present invention and CD16A-expressing cells of the present invention apply similarly to this description of pharmaceutical compositions and combination drugs of the present invention.

### VI. Methods of Preparing Modified Fc Regions of IgG, Modified Fcγ Receptors, and Combinations Thereof

The present invention also provides, in one aspect, a method for obtaining a polypeptide comprising a modified Fc region of IgG, the method comprising the steps of:
1) providing polypeptides comprising a modified Fc region of IgG, wherein the modified Fc region is non-naturally occurring and comprises at least one amino acid mutation compared to a wild type or naturally occurring IgG;
2) measuring the binding activity of the polypeptides obtained in 1) to a wild type or naturally occurring Fcγ receptor;
3) measuring the binding activity of the polypeptides obtained in 1) to a non-naturally occurring Fcγ receptor comprising at least one amino acid mutation compared to a wild type or naturally occurring Fcγ receptor; and
4) selecting from the polypeptides obtained in 1) a polypeptide having essentially no binding activity to a wild type or naturally occurring Fcγ receptor and which binds to the non-naturally occurring Fcγ receptor comprising at least one amino acid mutation.

In some embodiments, the polypeptide comprising a modified Fc region of IgG is an antibody. Step 4) may further include a step of contacting the antibody with an immune cell expressing the Fcγ receptor and a cell expressing an antigen to which the antibody binds, and then measuring ADCC activity.

The embodiments related to polypeptides of the present invention and CD16A-expressing cells of the present invention used in the method apply similarly to this description of a polypeptide of the present invention.

In another aspect, the present invention provides a method for preparing a non-naturally occurring Fcγ receptor. The method comprises the steps of:
1) providing non-naturally occurring Fcγ receptors comprising at least one amino acid mutation compared with a wild type or naturally occurring Fcγ receptor;
2) providing a polypeptide comprising an Fc region of wild type or naturally occurring IgG and a polypeptide comprising an Fc region of IgG comprising at least one amino acid mutation compared to the wild type or naturally occurring IgG;
3) measuring the binding activity of the non-naturally occurring Fcγ receptors obtained in 1) to polypeptide comprising the Fc region of the wild type or naturally occurring IgG;
4) measuring the binding activity of the non-naturally occurring Fcγ receptors obtained in 1) to the polypeptide comprising the Fc region of the IgG comprising at least one amino acid mutation; and
5) selecting from the non-naturally occurring Fcγ receptors obtained in 1) a non-naturally occurring Fcγ receptor having essentially no binding activity to the polypeptide comprising the Fc region of wild type or naturally occurring IgG and which binds to the polypeptide comprising the Fc region of the IgG comprising at least one amino acid mutation.

The embodiments related to polypeptides of the present invention and CD16A-expressing cells of the present invention used in the method apply similarly to this description of a polypeptide of the present invention.

In one aspect, the present invention provides a method for preparing a binding pair comprising (a) a polypeptide comprising a modified Fc region of IgG and (b) a non-naturally occurring modified Fcγ receptor. The method comprises the steps of:
1) providing a polypeptide comprising the Fc region of wild type or naturally occurring IgG and polypeptides comprising a modified Fc region of IgG, wherein the modified Fc region of the IgG is non-naturally occurring and comprises at least one amino acid mutation compared to the Fc region of the wild type or naturally occurring IgG;
2) providing a wild type or naturally occurring Fcγ receptor and non-naturally occurring modified Fcγ receptors, wherein the modified Fcγ receptors comprising at least one amino acid mutation compared to the wild type or naturally occurring Fcγ receptor;
3) measuring the binding activity of each Fcγ receptor obtained in 2) to each polypeptide obtained in 1); and
4) selecting (a) a polypeptide comprising a modified Fc region that binds to the modified Fcγ receptor and that has essentially no binding activity to the wild type or naturally occurring Fcγ receptor, and (b) a modified Fcγ receptor that binds to the polypeptide comprising the modified Fc region and that does not bind to the Fc region of the wild type or naturally occurring IgG.

In some embodiments, the polypeptide containing a modified Fc region of IgG is an antibody. Step 4) may further include a step of contacting the antibody with an immune cell expressing the Fcγ receptor and a cell expressing an antigen to which the antibody binds, and then measuring ADCC activity.

The embodiments related to polypeptides of the present invention and CD16A-expressing cells of the present invention used in the method apply similarly to this description of a polypeptide of the present invention.

In the methods described above, each step can be performed by those skilled in the art using any method known to those skilled in the art or using the methods described in the present specification.

Specific examples will now be provided for reference in order to further understand the present invention, but these are provided for illustrative purposes only and the present invention is not limited to these examples.

### EXAMPLES

There are two polymorphisms of the human CD16A: CD16A V158 and CD16A F158. The following examples were performed using CD16A V158 (“CD16V” below) which has higher binding activity to antibody Fc. The experiments using commercial kits or regents have been performed by following the attached protocol except when the method was demonstrated.

### Example 1: Protein Design Using In Silico Calculations

It has been reported by analyzing the three-dimensional structure of a complex protein that the binding activity and stability of a complex are affected by the introduction of a mutation into a charged amino acid that is considered important to complex formation, and that in silico calculations can be used to predict the effect of introducing mutations to charged amino acids on binding activity (Scientific Reports (2019) 9, pp. 4482). Therefore, the present inventors analyzed the three-dimensional structure of a complex between CD16V and antibody Fc (PDB code; 3ay4) using the MOE software (Chemical Computing Group), and extracted the basic or acidic amino acid residues on the binding interface between the CD16V and antibody Fc. Among these amino acid residues, a mutant was designed in which a basic amino acid on CD16V was replaced with an acidic amino acid or an acidic amino acid on the antibody Fc was replaced with a basic amino acid (Table 1). The names of the CD16V mutant described in the following example were in accordance with the number of the amino acid residues of the CD16A protein registered in 3ay4, which is based on the sequence of the CD16V protein (GenBank accession number: AAH17865.1) excluding the 1st to 18th amino acid sequences (SEQ ID NO: 78) (“3ay4” in the table). The mutations in the CD16V protein containing the 1st to 18th amino acid sequence corresponding to the mutations shown in “3ay4” are shown in the column “AAH17865.1” in Table 1.

The numbers under Fc in the table indicate the amino acid positions in the human Igγ1 constant region according to EU index numbering.

TABLE 1

| | |
|---|---|
| CD16V | Fc |

| Group | 3ay4 | AAH17865.1 | EU Index |
|---|---|---|---|
| Group 1 | K120D | K138D | D265K |
| | K120E | K138E | D265R |
| Group 2 | K128D | K146D | E294K |
| | K128E | K146E | E294R |
| Group 3 | K131D | K149D | E269K |
| | K131E | K149E | E269R |
| Group 4 | K161D | K179D | S239K |
| | K161E | K179E | S239R |

### Example 2: Preparation of a CD16V Protein and CD16V Mutant Proteins

In the present example, a protein with a mutation introduced to CD16V is denoted by “CD16V_introduced mutation” and referred to below collectively as CD16V mutant.

In order to obtain a CD16V protein, a gene encoding a polypeptide in the extracellular portion of CD16V (amino acids 1 to 208 in GenBank accession number: AAH17865.1) in which a FLAG sequence (DYKDDDDK, SEQ ID NO: 91) is linked to the C-terminus (SEQ ID NO: 1) was subcloned into a pcDNA3.4 vector (Thermo Fisher Scientific, Cat. A14697). The constructed vector was then transfected into ExpiCHO-S cells (Thermo Fisher Scientific, Cat. A29133). In order to obtain CD16V mutant proteins, a gene encoding a polypeptide in the extracellular portion of CD16V with an amino acid mutation shown in Table 1 (CD16V_K120D, CD16V_K120E, CD16V_K128D, CD16V_K128E, CD16V_K131D, CD16V_K131E, CD16V_K161D or CD16V_K161E) and a FLAG sequence linked to the C-terminus (SEQ ID NO: 3, 5, 7, 9, 11, 13, 15, or 17) was introduced to a pcDNA3.4 vector. The constructed vector was then transfected into ExpiCHO-S cells. The CD16V protein and the CD16V mutant protein were purified from the culture supernatant of ExpiCHO-S cells according to a standard method using anti-FLAG (registered trademark) M2 antibody affinity gel (SIGMA-ALDRICH, Cat. A2220). Note that the 1st to 18th amino acid sequences in the CD16V of AAH17865.1 are cleaved in mature form. The positions of the amino acid mutations introduced into the CD16V protein described in this example are in accordance with the amino acid numbers registered in 3ay4, and are numbered based on CD16V excluding the 1st to 18th amino acid sequence. Each mutation in the amino acid sequence of each CD16V mutant protein described in the sequence listing corresponds to a mutation shown in the “AAH17865.1” column of Table 1.

### Example 3: Production of an Fc_wt-Type Anti-HER2 Antibody or Mutant Fc-Type Anti-HER2 Antibodies

In the following study, trastuzumab (Drug Bank Accession Number: DB00072) was used as the anti-HER2 antibody.

Antibodies having the Fc sequence of the wild type human Igγ1 constant region are collectively referred to as Fc_wt. An expression vector used for production of a Fc_wt-type anti-HER2 antibody was constructed in the following manner. A heavy chain expression vector was constructed by inserting into a pcDNA3.4 vector a polynucleotide of a gene encoding the heavy chain variable region of trastuzumab (SEQ ID NO: 21) with a gene encoding a signal sequence (MEFGLSWVFLVAILKGVQC) (SEQ ID NO: 19) added to the 5′-side and a gene encoding the human Igγ1 constant region (SEQ ID NO: 23) added to the 3′-side. A light chain expression vector was constructed by inserting into a pcDNA3.4 vector a polynucleotide of a gene encoding the light chain region of trastuzumab (SEQ ID NO: 27) with a gene encoding a signal sequence (MDMRVPAQLLGLLLLWLRGARC) (SEQ ID NO: 25) added to the 5′-side. This light chain expression vector is referred to below as the trastuzumab light chain expression vector. These vectors were co-transfected into ExpiCHO-S cells, and an anti-HER2 antibody having a wild type Fc region (referred to below as the Fc_wt-type) was prepared from the culture supernatant according to a standard method.

Antibodies having a mutation in the Fc region (referred to below as “Fc_introduced mutations” and referred to below collectively as mutant Fc-type antibodies) were prepared.

Heavy chain expression vectors used in the production of Fc_S239K, Fc_S239R, Fc_E294K, and Fc_E294R were constructed by introducing into pcDNA3.4 vectors a polynucleotide of a gene encoding the heavy chain variable region of trastuzumab (SEQ ID NO: 21) with a gene encoding a signal sequence (MEFGLSWVFLVAILKGVQC) (SEQ ID NO: 19) added on the 5′-side and with a gene encoding a human Igγ1 constant region into which amino acid mutations for substituting lysine (K) or arginine (R) at S239 or E294 have been introduced (SEQ ID NOs: 33, 35, 29 and 31) added on the 3-'side. Heavy chain expression vectors used in the production of Fc_D265K, Fc_D265R, Fc_E269K, and Fc_E269R were constructed by introducing into pcDNA3.4 vectors a polynucleotide of a gene encoding the heavy chain variable region of trastuzumab (SEQ ID NO: 39) with a gene encoding a signal sequence (MEWSWVFLFFLSVTTGVHS) (SEQ ID NO: 37) added on the 5′-side and with a gene encoding a human Igγ1 constant region into which amino acid mutations for substituting lysine (K) or arginine (R) at D265 or E269 have been introduced (SEQ ID NOs: 41, 43, 45 and 47) added on the 3-'side. Each one of these expression vectors was co-transfected with the trastuzumab light chain expression vector into ExpiCHO-S cells, and mutant Fc-type anti-HER2 antibodies were prepared from the culture supernatant according to a standard method.

### Example 4: Evaluation of the Binding Activity of Fc_wt-Type and Mutant Fc-Type Anti-HER2 Antibodies With CD16V and CD16V Mutant Proteins

The binding activity of the CD16V and CD16V mutants obtained in Example 2 with the Fc_wt-type and mutant Fc-type anti-HER2 antibodies obtained in Example 3 was evaluated. A HER2 protein (Sino Biological, Cat. 10004-H08H) was diluted to 2 μg/mL with phosphate buffered saline (PBS), 20 μL of the diluted HER2 protein was added to each well of a Maxisorp 384-well transparent plate (Thermo Fisher Scientific, Cat. 464718), and immobilized by overnight incubation at 4° C. After the HER2 protein solution was removed, the plate was incubated with 50 μL of PBS containing Blocking One (Nacalai Tesque, Cat. 03953-95) for one hour at room temperature, and then washed with Tris buffered saline containing 0.05% Tween 20 (TBS-T, Nippon Gene, Cat. 310-07375). Each anti-HER2 antibody obtained in Example 3 was diluted to 4μg/mL with TBS-T containing 5% Blocking One (referred to below simply as the diluent), and 20 μL of the diluted antibody was added to each well. After incubation at room temperature for one hour, the plate was washed with TBS-T. Each CD16V protein obtained in Example 2 was diluted with the diluent to prepare a dilution series at about a three-fold common ratio from a maximum concentration of 10 μg/mL, and 20 μL of the diluted CD16V protein was added to each well. After incubation at room temperature for one hour, the plate was washed with TBS-T. Next, 20 μL of horseradish peroxidase-labeled anti-FLAG (registered trademark) M2 antibody (SIGMA-ALDRICH, Cat. A8592) diluted with the diluent was added to each well as a detection antibody. After incubating at room temperature for one hour, the plate was washed with TBS-T. After adding TMB+Substrate-Chromogen (DAKO, Cat. S1599) and incubating, the reaction was stopped by adding 1M sulfuric acid, and the absorbance at 450 nm and reference wavelength were measured using Infinite 200 PRO (TECAN).

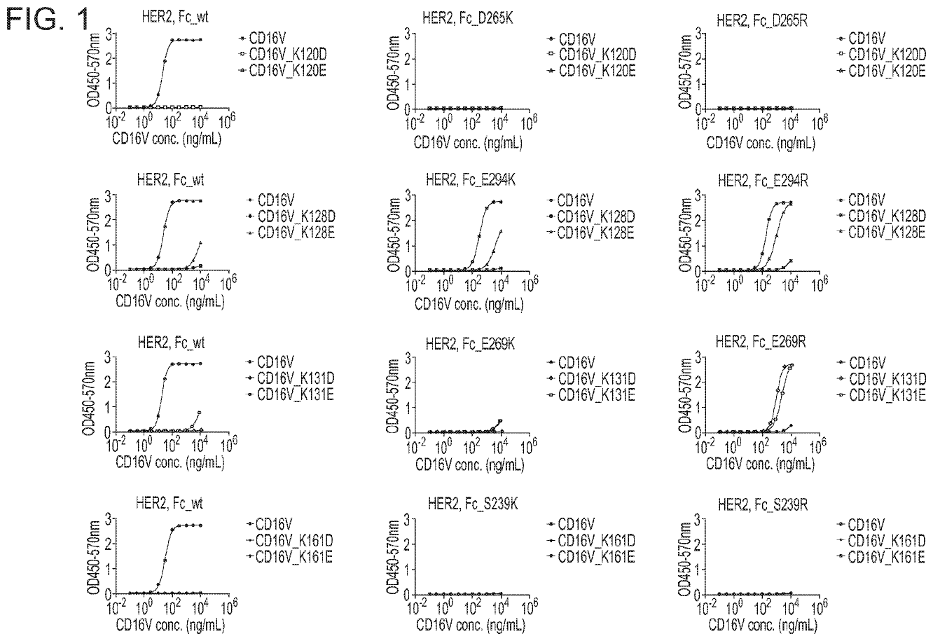

As a result, CD16V_K131D did not bind to the Fc_wt, but only to Fc_E269R. However, the binding activity between CD16V_K131D and Fc_E269R was lower than the binding activity between Fc_wt and CD16V. Also, Fc_E269R did bind slightly to CD16V. While it was confirmed that both CD16V_K128E and K131E did bind to mutant Fc, binding to Fc_wt was only somewhat confirmed. Meanwhile, no binding or extremely low binding of CD16V_K128D, CD16V_K120D, CD16V_K120E, CD16V_K161D and CD16V_K161E with Fc_wt or any of the mutant Fc was confirmed (FIG. 1).

This suggests that position K128 and position K131 in CD16V as well as position E269 and position E294 in the Fc region of the antibodies are important amino acid residues for specific binding activity between CD16V mutants and mutant Fc type antibodies. Therefore, a study was conducted to obtain mutant Fc and CD16V mutants that only binds specifically each other into which specific amino acid mutations have been introduced using combinations of these mutations and combinations of these mutations with mutations known to increase binding activity.

### Example 5: Production of Mutant Fc-Type Anti-HER2 Antibodies and CD16V Mutant Proteins

CD16V_K128E_K131D (referred to as CD16V_ED below) and CD16V_K128E_K131E (referred to as CD16V_EE below) were prepared by combining mutations of K128E and K131D or K131E with CD16V. Specifically, as in Example 2, an expression vector was constructed by introducing to a pcDNA3.4 vector a gene encoding an extracellular polypeptide of CD16V protein with these amino acid mutations linked to a FLAG sequence (SEQ ID NOs: 49 and 51) introduced at the C-terminus. This expression vector was then transfected into ExpiCHO-S cells. CD16V_ED and CD16V_EE were prepared from the culture supernatant of each in the same manner as Example 2.

It has been reported that mutagenesis of N38Q, N74Q or N169Q into CD16V slightly enhances the binding activity of Fc_wt (Journal of Biological Chemistry (2018) 293, pp. 16842-16850). CD16V_K131D (referred to below as CD16V_D) bound only to the Fc_E269R-type antibody, therefore, CD16V_K131D_N38Q (referred to below as CD16V_DQ1) and CD16V_K131D_N74Q (referred to below as CD16V_DQ2) in which CD16V_D is combined with N38Q or N74Q mutations were prepared. Specifically, expression vectors were constructed in the same manner as Example 2 by introducing into a pcDNA3.4 vector a gene encoding a polypeptide of an extracellular domain of the CD16V protein, in which one of these amino acid mutations is introduced, linked to a FLAG sequence (SEQ ID NOs: 53 and 55) is introduced to the C-terminal. Each expression vector was then transfected into ExpiCHO-S cells. CD16V_DQ1 and CD16V_DQ2 were prepared from the culture supernatant of each in the same manner as Example 2.

Fc_E269R_E294K (referred to below as Fc_RK) and Fc_E269R_E294R (referred to below as Fc_RR) type anti-HER2 antibodies were prepared. Specifically, expression vectors were constructed in the same manner as Example 3 by introducing into a pcDNA3.4 vector a polynucleotide of a gene encoding the heavy chain variable region of trastuzumab (SEQ ID NO: 39) with a gene encoding a signal sequence (MEWSWVFLFFLSVTTGVHS) (SEQ ID NO: 37) added to the 5′-side and with a gene encoding a human Igγ1 constant region having amino acid mutations introduced to arginine at position E269 and to lysine (K) or arginine (R) at position E294 (SEQ ID NOs: 57 and 59) added to the 3′-side. The resulting heavy chain expression vector and the trastuzumab light chain expression vector obtained in Example 3 were co-transfected to ExpiCHO-S cells, and mutant Fc-type anti-HER2 antibodies were prepared from the culture supernatant of each according to a standard method.

### Example 6: Evaluation of the Binding Activity of Fc_wt-Type and Mutant Fc-Type Anti-HER2 Antibodies With CD16V and CD16V Mutant Proteins

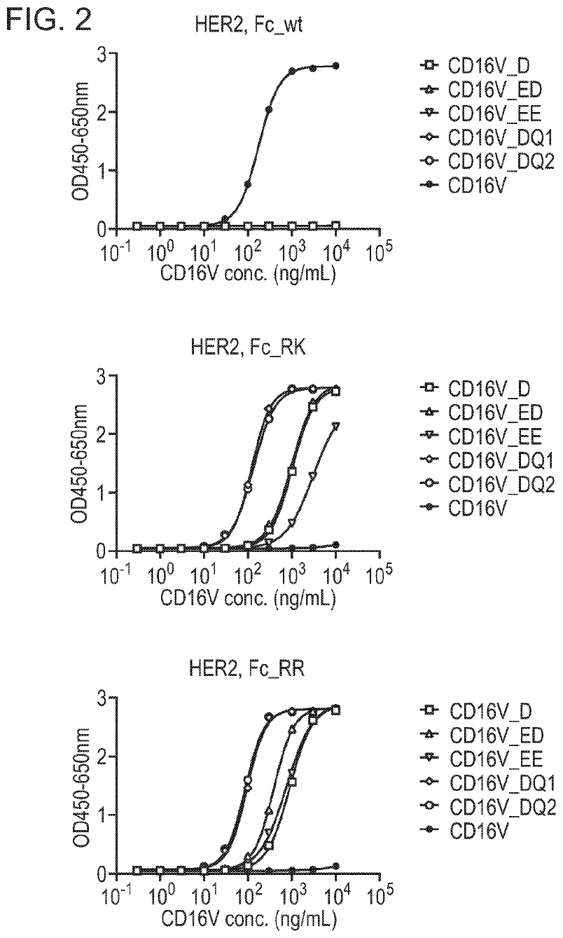

The binding activity of CD16V_D obtained in Example 2 and CD16V_ED, CD16V_EE, CD16V_DQ1 and CD16V_DQ2 obtained in Example 5 with Fc_wt type anti-HER2 antibody obtained in Example 3, or Fc_RK or Fc_RR type anti-HER2 antibodies obtained in Example 5 was evaluated in the same manner as Example 4 (FIG. 2).

As a result, the Fc_wt type anti-HER2 antibody bound to CD16V but did not exhibit binding activity to CD16V_D, CD16V_ED, CD16V_EE, CD16V_DQ1 and CD16V_DQ2. Meanwhile, the Fc_RK or Fc_RR type anti-HER2 antibodies bound to the CD16V mutant but not to CD16V. Therefore, it is clear that the binding specificity of the mutant Fc-type antibody to the CD16V mutant was enhanced by introducing a mutation at position E294 into Fc_E269R. Also, the binding activity of CD16V_DQ1 and CD16V_DQ2 to Fc_RK and Fc_RR was almost the same as the binding activity of CD16V to Fc_wt. Therefore, it is clear that CD16V_DQ1 and CD16V_DQ2, in which additional mutations are introduced into CD16V_D, do not have enhanced Fc_wt binding activity but only have enhanced Fc_RR and Fc_RK binding activity.

### Example 7: Production of Fc_wt-Type and Mutant Fc-Type Anti-EGFR Antibodies and Anti-EpCAM Antibodies

Fc_wt, Fc_RK, and Fc_RR type anti-EGFR antibodies and anti-EpCAM antibodies were prepared in order to examine whether the Fc sequence prepared in Example 5 had specific binding activity for CD16V_D obtained in Example 2 and CD16V_ED, CD16V_EE, CD16V_DQ1 and CD16V_DQ2 obtained in Example 5 when used in antibodies other than anti-HER2 antibodies.

In production of the Fc_wt, Fc_RK, and Fc_RR type anti-EGFR antibodies, a heavy chain expression vector was prepared by introducing into a pcDNA3.4 vector a polynucleotide of the gene encoding the heavy chain variable region of anti-EGFR (cetuximab, Drug Bank Accession No: DB00002) (SEQ ID NO: 63) with a gene encoding a signal sequence (MEFGLSWVFLVALLRGVQC) (SEQ ID NO: 61) added to the 5′-side, and with a gene encoding the human Igγ1 constant region (SEQ ID NO: 23), a gene encoding a human Igγ1 constant region containing Fc_RK (SEQ ID NO: 57), or a gene encoding a human Igγ1 constant region including Fc_RR (SEQ ID NO: 59) added to the 3′-side. Also, a light chain expression vector was prepared by introducing into a pcDNA3.4 vector a gene encoding the light chain region of cetuximab (SEQ ID NO: 67) with a gene encoding the signal sequence (MLPSQLIGFLLLWVPASRG) (SEQ ID NO: 65) added to the 5′-side.

In production of the Fc_wt, Fc_RK, and Fc_RR type anti-EpCAM antibodies, a heavy chain expression vector was prepared by introducing into a pcDNA3.4 vector a polynucleotide of the gene encoding the heavy chain variable region of anti-EpCAM (edrecolomab, IMGT INN No: 7471) (SEQ ID NO: 71) with a gene encoding a signal sequence (MEWSWVFLFFLSVTTGVHS) (SEQ ID NO: 69) added to the 5′-side, and with a gene encoding the human Igγ1 constant region (SEQ ID NO: 23), a gene encoding a human Igγ1 constant region containing Fc_RK (SEQ ID NO: 57), or a gene encoding a human Igγ1 constant region including Fc_RR (SEQ ID NO: 59) added to the 3′-side. Also, a light chain expression vector was prepared by introducing into a pcDNA3.4 vector a gene encoding the light chain region of edrecolomab (SEQ ID NO: 75) with a gene encoding the signal sequence (MSVPTQVLGLLLLWLTDARC) (SEQ ID NO: 73) added to the 5′-side.

Each one of the heavy chain expression vectors was co-transfected with the cetuximab light chain or the edrecolomab light chain expression vector into ExpiCHO-S cells, and anti-EGFR antibodies and anti-EpCAM antibodies were purified in the same manner as Example 3.

### Example 8: Evaluation of the Binding Activity of Fc_wt-Type and Mutant Fc-Type Anti-EGFR Antibodies or Anti-EpCAM Antibodies With CD16V and CD16V Mutant Proteins

The binding activity of the CD16V, CD16V_D obtained in Example 2 and CD16V_ED, CD16V_EE, CD16V_DQ1 and CD16V_DQ2 obtained in Example 5 with the Fc_wt, Fc_RK or Fc_RR type anti-EGFR antibodies or anti-EpCAM antibodies obtained in Example 7 was evaluated. An EGFR protein (Abcam, Cat. ab155639) was diluted to 4 μg/mL or EpCAM protein (Sino Biological, Cat. 10694-H08H) was diluted to 2 μg/mL in PBS, 20 μL of the diluted protein was added to each well of a Maxisorp 384-well transparent plate, and immobilized by overnight incubation at 4° C. The next day, the EGFR or EpCAM protein solution was removed, the plate was incubated with 50 μL of PBS containing Blocking One for one hour at room temperature, and then washed with TBS-T. Each anti-EGFR antibody obtained in Example 7 was diluted to 2 μg/mL with the diluent, each anti-EpCAM antibody obtained in the same example was diluted to 10 μg/mL with the diluent, and 20 μL of the diluted antibody was added to each well. After incubation at room temperature for one hour, the plate was washed with TBS-T. Each CD16V protein obtained in Example 2 or Example 5 was diluted with the diluent to prepare a dilution series at about a three-fold common ratio from a maximum concentration of 10 μg/mL, and 20 μL of the diluted CD16V protein was added to each well. After incubation at room temperature for one hour, the plate was washed with TBS-T. Next, 20 μL of horseradish peroxidase-labeled anti-FLAG (registered trademark) M2 antibody diluted with the diluent was added to each well as a detection antibody. After incubating at room temperature for one hour, the plate was washed with TBS-T. After adding TMB+Substrate-Chromogen and incubating, the reaction was stopped by adding 1M sulfuric acid, and the absorbance at 450 nm and reference wavelength were measured using Infinite 200 PRO.

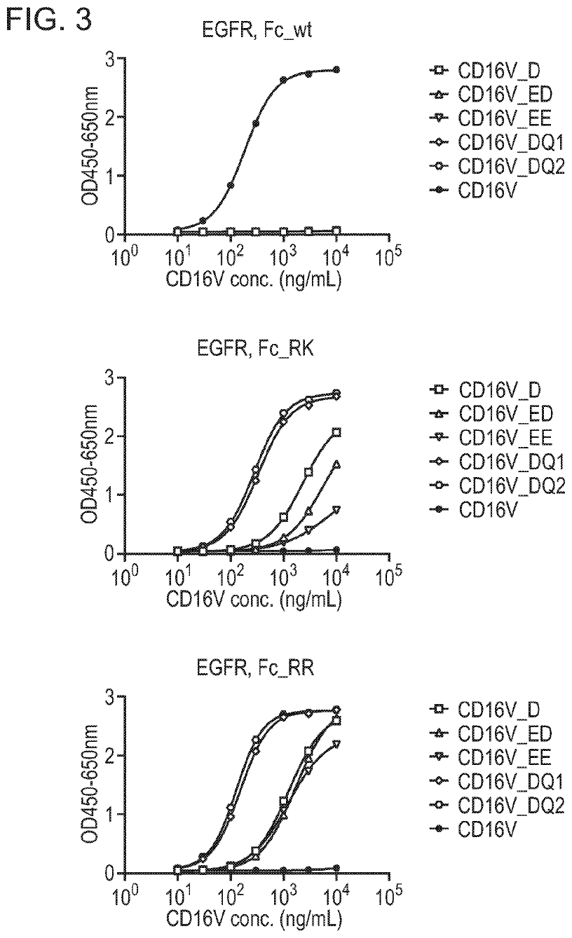

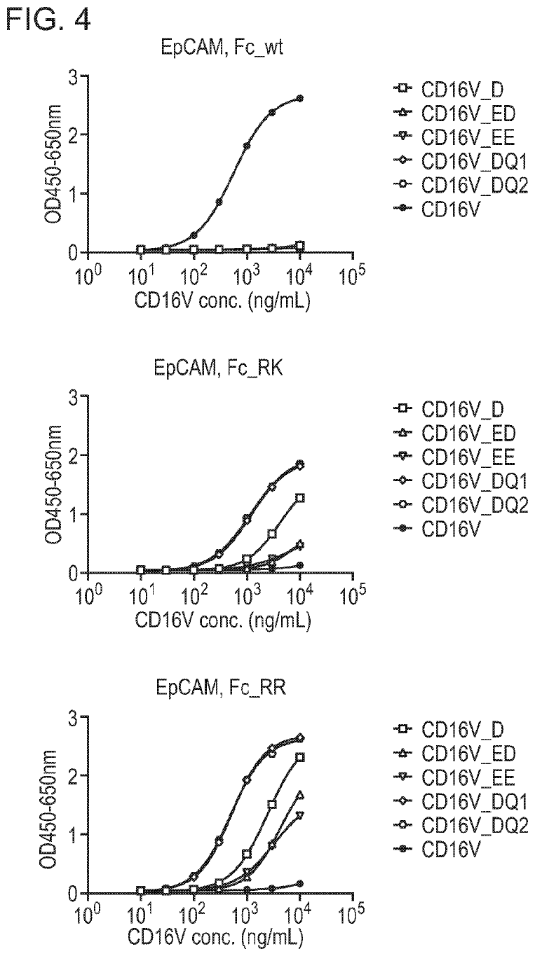

In the case of the anti-EGFR antibodies and the anti-EpCAM antibodies, as in the case of the anti-HER2 antibodies, the Fc_wt type antibody bound to CD16V but did not bind to the CD16V mutants, and the Fc_RK and Fc_RR type antibodies did not bind to CD16V but only to the CD16V mutants (FIG. 3, FIG. 4). It is clear from these results that Fc_RK and Fc_RR not only in anti-HER2 antibodies but also in other antibodies bind specifically to CD16V mutants.

### Example 9: Evaluation of the Binding Activity of Mutant Fc-Type Anti-HER2 Antibodies With CD16V and CD16V Mutant Proteins in the Presence of Excess IgG1 Antibodies

A competition test using Fc_wt type antibody was performed in order to confirm the binding activity between mutant Fc-type anti-HER2 antibodies and CD16V mutant proteins in vivo. The Fc_wt type antibody used here as mimicking endogenous immunoglobulin was the human IgG1 antibody against keyhole limpet hemocyanin (KLH), an antigen that does not exist in vivo. This antibody was obtained according to a standard method. As in Example 4, a HER2 protein was immobilized on a Maxisorp 384-well transparent plate. The next day, after the HER2 protein solution was removed, the plate was incubated with PBS containing Blocking One for one hour at room temperature, and then was washed with TBS-T. Fc_wt, Fc_RK and Fc_RR type anti-HER2 antibodies were added to each well and incubated at room temperature for one hour. Each CD16V and CD16V mutant protein was diluted with the diluent to prepare a dilution series at about a three-fold common ratio from a maximum concentration of 20 μg/mL, and mixed at 1:1 with the diluent or anti-KLH antibodies adjusted to 2 mg/mL with the diluent. After the plate was washed with TBS-T, 20 μL of these mixed solutions were added and incubated at room temperature for one hour. At this time, there was about a three-fold common ratio from a CD16V protein final concentration of 10 μg/mL. After the plate was washed with TBS-T, 20 μL of horseradish peroxidase-labeled anti-FLAG (registered trademark) M2 antibody diluted by a factor of 2,000 with the diluent was added to each well as a detection antibody. After incubating at room temperature for one hour, the plate was washed with TBS-T. After adding TMB+Substrate-Chromogen and incubating, the reaction was stopped by adding 1M sulfuric acid, and the absorbance at 450 nm and reference wavelength were measured using Infinite 200 PRO.

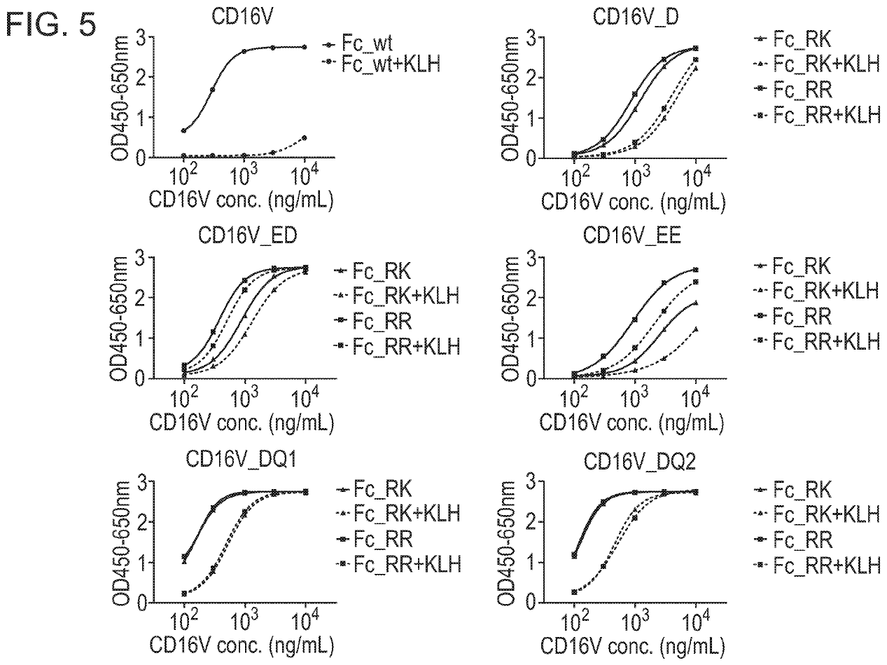

As a result, the binding activity between CD16V and Fc_wt type anti-HER2 antibodies in the presence of the anti-KLH antibody was very low at the maximum CD16V concentration of 10 μg/mL, and the absorbance when CD16V was reacted at 3 μg/mL or less was at the same level as the background. Meanwhile, the binding activity of the Fc_RK and Fc_RR type anti-HER2 antibodies with each of the CD16V mutants (CD16V_D, CD16V_ED, CD16V_EE, CD16V_DQ1 and CD16V_DQ2) in the presence of the anti-KLH antibody decreased but remained within a certain range of decline (FIG. 5). Because most of the immunoglobulin in serum is IgG1, this suggests that mutant Fc-type antibodies and CD16V mutants exhibit binding at lower concentrations than Fc_wt and CD16V in serum.

### Example 10: Establishment of CD16V-Expressing KHYG-1 Cells

The CD16V sequence is known to have a mature type (SEQ ID NO: 78) and an immature type (GenBank accession number: AAH17865.1, SEQ ID NO: 90). A gene encoding the immature type (SEQ ID NO: 89) was inserted into a multicloning site in a pLVSIN-CMV Pur Vector (Takara Bio, Cat. 6183). Similarly, genes (SEQ ID NOs: 79, 81, 83, 85, 87) encoding the amino acid sequences of CD16V mutants (CD16V_D, CD16V_ED, CD16V_EE, CD16V_DQ1, CD16V_DQ2) (SEQ ID NOs: 80, 82, 84, 86, 88) were introduced into a pLVSIN-CMV Pur Vector to construct vectors of each CD16V mutant. The Lenti-X (registered trademark) 293T Cell Line (Takara Bio, Cat. 632180) was transfected with these vectors using Lipofectamine 2000 (Thermo Fisher Scientific, Cat. 11668027) with Lentiviral High Titer Packaging Mix (Takara Bio, Cat. 6194), and the resulting culture supernatants were collected. PEG-it Virus Precipitation Solution (5×) (System Biosciences, Cat. LV810A-1) was added to each culture supernatant and incubated overnight at 4° C. to concentrate CD16V or CD16V mutant-expressing lentiviral vectors. By infecting the lentiviral vectors into KHYG-1 cells (JCRB Cell Bank, JCRB0156) and selecting only transgenic cells with Puromycin, CD16V-expressing KHYG-1 (CD16V/KHYG-1) cells or CD16V mutant-expressing KHYG-1 (CD16V mutant/KHYG-1) cells with introduced CD16V or CD16V mutant genes were prepared.

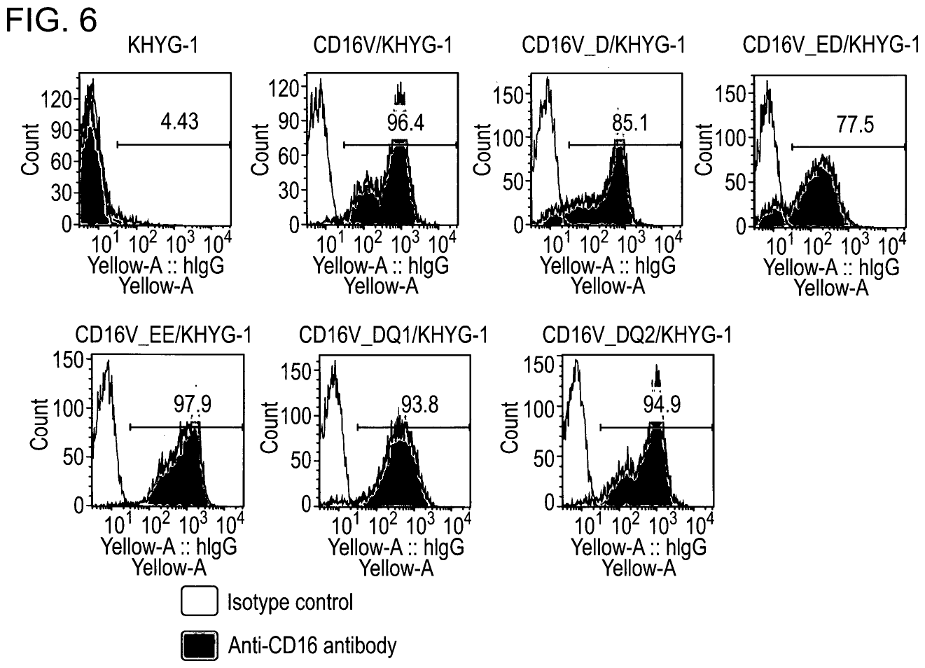

The expression level of CD16V or CD16V mutant expressed in the prepared CD16V/KHYG-1 cells or CD16V mutant/KHYG-1 cells was measured by flow cytometry. Phycoerythrin-labeled anti-human CD16 antibodies (clone: 3G8, BioLegend, Cat. 302008) were added to each KHYG-1 cell suspended in STAIN BUFFER (BD Bioscience, Cat. 554656), and incubated for 20 minutes on ice. After washing the cells three times with STAIN BUFFER, 7-AAD (BD Bioscience, Cat. 559925) was added and incubated for 15 minutes in a dark condition. The fluorescence intensity of phycoerythrin was then measured in the 7-AAD negative viable cell fraction using a FACS Array (BD Bioscience). FlowJo (BD Bioscience) was used for the analysis. As a result, all of the established cells expressed CD16V or a CD16V mutant at an expression rate of 75% or more (FIG. 6).

### Example 11: Evaluation of Antibody ADCC Activity Using CD16V-Expressing KHYG-1 Cells

In order to evaluate the ADCC activity of CD16V/KHYG-1 cells or CD16V mutant/KHYG-1 cells, ten thousand cells of HER2-positive SK-BR-3 cells stained with Calcein-AM Solution (Dojindo Laboratories, C396) and one hundred thousand cells of CD16V/KHYG-1 cells or CD16V mutant/KHYG-1 cells at 1:10 ratio were incubated for four hours in the presence of each anti-HER2 antibody diluted in a four-fold dilution series from the highest concentration of 4 μg/mL in 200 μL of culture medium in a 96-well plate (CORNING, Cat. 353077). The fluorescence intensity derived from Calcein-AM in 100 μL of the supernatant was measured using the FlexStation 3 (Molecular Devices) to observe the SK-BR-3 cell viability. The cytotoxic activity was calculated using the following formula where T-MAX is the value when 1% Triton X-100 (SIGMA-ALDRICH, Cat. 30-5140) was added to the SK-BR-3 cells, and T-Spon is the value when only the culture medium was added.

Cytotoxic Activity (%) (% of Lysis)=100×(Data−(T-Spon))/((T-MAX)−(T-Spon))

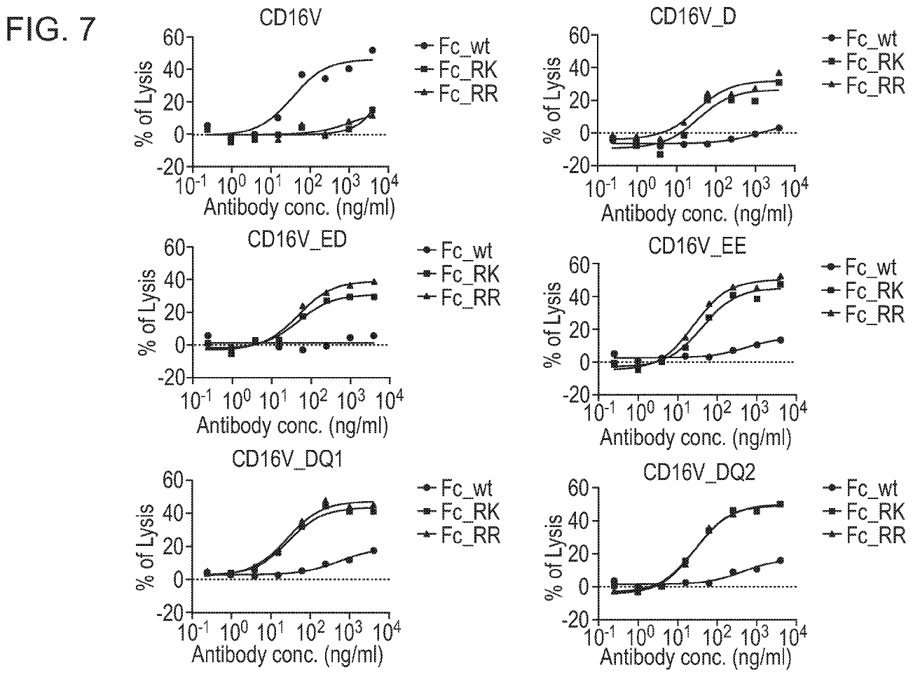

As a result, the CD16V/KHYG-1 cells exhibited cytotoxicity against SK-BR-3 cells in a concentration-dependent manner only in the case of the Fc_wt type anti-HER2 antibodies. The cytotoxicity was extremely low in the case of the Fc_RK and Fc_RR type anti-HER2 antibodies. Meanwhile, the Fc_wt type anti-HER2 antibodies did not exhibit cytotoxic activity or exhibited extremely low cytotoxic activity in all of the CD16V mutant/KHYG-1 cells that were evaluated. Only Fc_RK and Fc_RR type anti-HER2 antibodies exhibited cytotoxic activity in all of the CD16V mutant/KHYG-1 cells that were evaluated (FIG. 7). Therefore, it is clear that CD16V mutants and Fc mutants have specific binding activity and combinations exhibit specificity in ADCC inducing action.

Example 12: Evaluation of ADCC Activity Using CD16V-Expressing KHYG-1 Cells in the Presence of Human Serum

In order to identify the effect of human serum on ADCC activity of CD16V or CD16V mutant/KHYG-1 cells, the cytotoxicity against SK-BR-3 cells was evaluated using each anti-HER2 antibody at concentration of 1 μg/mL in culture medium containing 5% fetal bovine serumFBS or 5% human serum in the same manner as Example 11. The complement activity of each serum was heat-inactivated to avoid interference with evaluation of ADCC activity by induction of complement-dependent cytotoxicity.

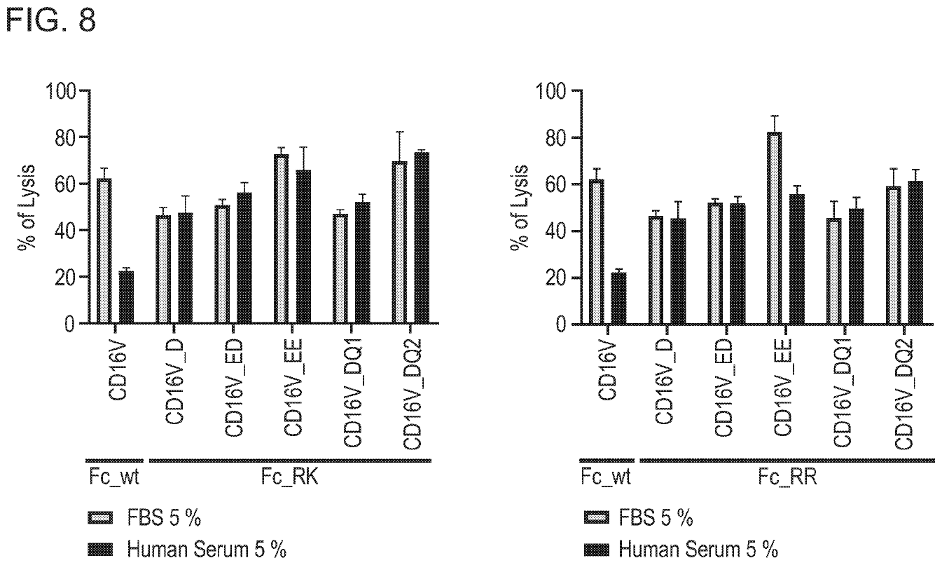

As a result, the cytotoxicity against SK-BR-3 of CD16V/KHYG-1 in the case of Fc_wt type anti-HER2 antibody decreased in the presence of human serum compared with FBS. Meanwhile, the cytotoxicity against SK-BR-3 of CD16V mutant/KHYG-1 in the case of Fc_RK type anti-HER2 antibody in the presence of human serum was almost the same as in the presence of FBS (FIG. 8). Similarly, the cytotoxicity against SK-BR-3 of CD16V mutant/KHYG-1 in the case of Fc_RR type anti-HER2 antibody in the presence of human serum was almost the same as in the presence of FBS except for CD16V_EE/KHYG-1 (FIG. 8).

Therefore, this result indicates that human serum interferes with the interaction between Fc_wt type antibody and CD16V, while Fc_RK or Fc_RR type antibody and human serum IgG have less competition for ADCC inducing action on CD16V mutants/KHYG-1.

### Example 13: Evaluation of Cytotoxic Activity of CD16V CAR-Expressing T Cells

CAR-T is known to be one of the most powerful effector cells against cancer cells in cancer immunotherapy. To expand applications for the combination of CD16V mutants and Fc mutants, chimeric receptor in which extracellular domain of CD16V or CD16V mutant fused with signal transduction domain (CD16V CAR or CD16V mutant CAR) was expressed on primary T cells, and cytotoxic activity of these CD16V CAR or CD16V mutants CAR-expressing T cells (CD16V/CAR-T or CD16V mutants/CAR-T) against cancer cells was evaluated.

A gene encoding the amino acid sequence of CD16V CAR (SEQ ID NO: 92), which is the fusion protein of the extracellular domain of CD16V and signal domain of CD3ζ and CD137 via CD8a hinge and CD8 transmembrane domain with a gene encoding a signal sequence (MALPVTALLLPLALLLHAARP) (SEQ ID NO: 104) added to the 5′-side, was inserted into a multicloning site in a pLVSIN-EF1α Neo Vector (Takara Bio, Cat. 6184). Similarly, each of genes (SEQ ID NOs: 94, 96, 98, 100, and 102) encoding the amino acid sequences of each CD16V mutant (CD16V_D, CD16V_ED, CD16V_EE, CD16V_DQ1, and CD16V_DQ2) CAR with a gene encoding a signal sequence (MALPVTALLLPLALLLHAARP) (SEQ ID NO: 104) added to the 5′-side was inserted into a multicloning site in a pLVSIN-EF1α Neo Vector. Using these vectors, CD16V or CD16V mutant-expressing lentiviral vectors were prepared in the same manner as Example 10.

By infecting the lentiviral vectors into human T cells purified from human peripheral blood mononuclear cells (Lonza, Cat. CC-2702) using Pan T cells isolation kit (Miltenyi Biotec, Cat. 130-096-535), CD16V/CAR-T and CD16V mutant/CAR-T were prepared. The expression rate of CD16V or each CD16V mutant on T cells were over 60%. In order to evaluate the cytotoxic activity of CD16V/CAR-T or CD16V mutant/CAR-T, ten thousand cells of SK-BR-3 cells stained with Calcein-AM Solution and one hundred and fifty thousand cells of CD16V/CAR-T or CD16V mutant/CAR-T at 1:15 ratio were incubated for four hours in the presence of each anti-HER2 antibody diluted in a four-fold dilution series from the highest concentration of 16 μg/mL in 200 μL of culture medium. The fluorescence intensity derived from Calcein-AM in 100 μL of the supernatant was measured and the cytotoxic activity was calculated in the same manner as Example 11.

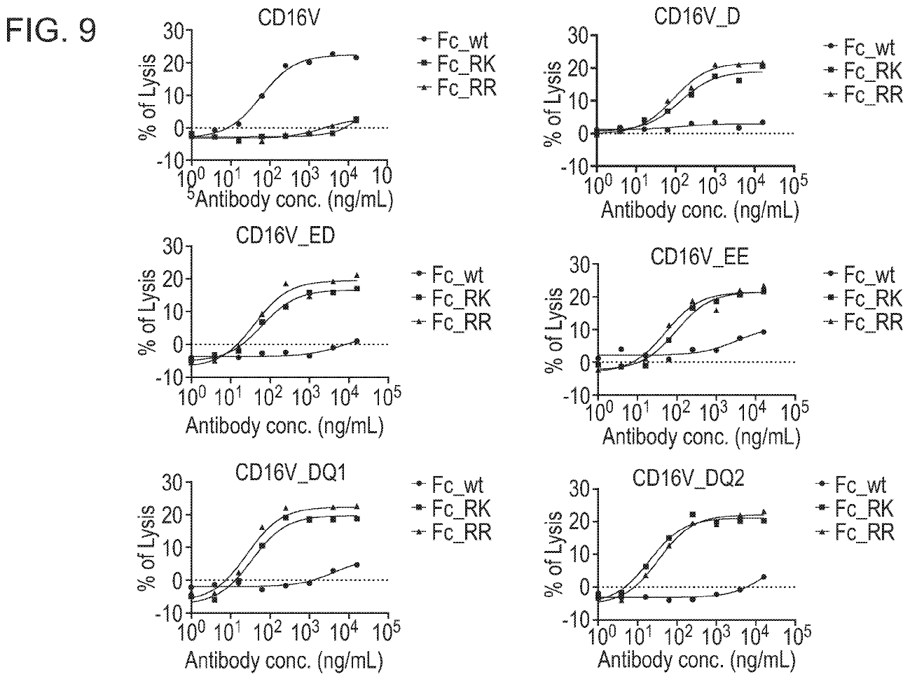

As a result, the CD16V/CAR-T exhibited cytotoxicity against SK-BR-3 cells in a concentration-dependent manner only in the case of the Fc_wt type anti-HER2 antibodies. The cytotoxicity was extremely low in the case of the Fc_RK and Fc_RR type anti-HER2 antibodies. Meanwhile, the Fc_wt type anti-HER2 antibodies did not induce cytotoxic activity or induced extremely low cytotoxic activity of all CD16V mutants/CAR-T. Only Fc_RK and Fc_RR type anti-HER2 antibodies induced cytotoxic activity of all CD16V mutants/CAR-T (FIG. 9). Therefore, it is clear the combinations of CD16V mutants and Fc mutants keeps the specificity in the case of application to chimeric protein like CAR.

The Fcγ receptor mutants and the Fc region mutants of the present invention do not essentially bind to endogenous immunoglobulin or endogenous Fcγ receptors, but the Fcγ receptor mutants and the Fc region mutants specifically bind to each other. Therefore, these combinations are expected to provide an immunotherapy in which endogenous molecules do not diminish drug efficacy.

### Equivalents

Those skilled in the art will recognize, or be able to ascertain using no more than routine experimentation, many equivalents to the specific embodiments and methods described herein. Such equivalents are intended to be encompassed by the scope of the following claims.

### [Sequence Listing Free Text]

A description of the Artificial Sequence is provided under the numerical heading <223> in the sequence listing below. Specifically, the nucleotide sequence represented by SEQ ID NO: 1 in the sequence listing is a nucleotide sequence encoding a protein in which a FLAG sequence is linked to the C-terminus of an extracellular partial sequence protein of the CD16V sequence. The amino acid sequence shown in SEQ ID NO: 2 is the amino acid sequence encoded by SEQ ID NO: 1. The nucleotide sequences shown in SEQ ID NOs: 3, 5, 7, 9, 11, 13, 15 and 17 are nucleotide sequences encoding a protein in which a FLAG sequence is linked to the C-terminus of a protein in which a mutation has been introduced into an extracellular subsequence of the CD16V sequence. The amino acid sequences shown in SEQ ID NOs: 4, 6, 8, 10, 12, 14, 16 and 18 are amino acid sequences encoded by SEQ ID NOs: 3, 5, 7, 9, 11, 13, 15 and 17, respectively. SEQ ID NOs: 19 and 37 are nucleotide sequences encoding a signal sequence linked to the N-terminus of a gene encoding a heavy chain variable region of trastuzumab. SEQ ID NOs: 21 and 39 are nucleotide sequences encoding a heavy chain variable region of trastuzumab, and SEQ ID NO: 23 is a nucleotide sequence encoding the human Igγ1 constant region. SEQ ID NOs: 20, 22, 24, 38 and 40 are the amino acid sequences encoded by SEQ ID NOs: 19, 21, 23, 37 and 39, respectively. SEQ ID NO: 27 is a nucleotide sequence encoding a light chain region of trastuzumab, and SEQ ID NO: 28 is an amino acid sequence encoded by SEQ ID NO: 27. SEQ ID NO: 25 is a nucleotide sequence encoding a signal sequence linked to the 5′-side of a light chain region of trastuzumab, and SEQ ID NO: 26 is an amino acid sequence encoded by SEQ ID NO: 25. SEQ ID NOs: 29, 31, 33, and 35 are nucleotide sequences encoding proteins with mutations introduced into a gene encoding the human Igγ1 constant region, and SEQ ID NOs: 30, 32, 34, and 36 are the amino acid sequences encoded by SEQ ID NOs: 29, 31, 33, and 35, respectively. The nucleotide sequences represented by SEQ ID NOs: 41, 43, 45 and 47 are nucleotide sequences encoding a protein with amino acid mutations introduced into a gene encoding the human Igγ1 constant region, and SEQ ID NOs: 42, 44, 46 and 48 are the amino acid sequences encoded by SEQ ID NOs: 41, 43, 45 and 47, respectively. The nucleotide sequences shown in SEQ ID NOs: 49, 51, 53 and 55 are nucleotide sequences encoding a protein in which a FLAG sequence is linked to the C-terminus of a protein in which mutations have been introduced into an extracellular subsequence of the CD16V sequence. The amino acid sequences shown in SEQ ID NOs: 50, 52, 54 and 56 are the amino acid sequences encoded by SEQ ID NOs: 49, 51, 53 and 55, respectively. The nucleotide sequences represented by SEQ ID NOs: 57 and 59 are nucleotide sequences encoding a protein having mutations introduced into the human Igγ1 constant region. SEQ ID NOs: 58 and 60 are the amino acid sequences encoded by SEQ ID NOs: 57 and 59. SEQ ID NO: 61 is a nucleotide sequence encoding a signal sequence linked to the N-terminus of a gene encoding a heavy chain variable region of cetuximab, and SEQ ID NO: 65 is a nucleotide sequence encoding a signal sequence linked to the N-terminus of a gene encoding a light chain region of cetuximab. SEQ ID NOs: 62 and 66 are the amino acid sequences encoded by SEQ ID NOs: 61 and 65. SEQ ID NOs: 63 and 67 are nucleotide sequences encoding a heavy chain variable region and a light chain region of cetuximab, respectively, and SEQ ID NOs: 64 and 68 are the amino acid sequences encoded by SEQ ID NOs: 63 and 67, respectively. SEQ ID NO: 69 is a nucleotide sequence encoding a signal sequence linked to the N-terminus of a gene encoding a heavy chain variable region of edrecolomab, and SEQ ID NO: 73 is a nucleotide sequence encoding a signal sequence linked to the N-terminus of a gene encoding a light chain region of edrecolomab. SEQ ID NOs: 70 and 74 are the amino acid sequences encoded by SEQ ID NOs: 69 and 73, respectively. SEQ ID NOs: 71 and 75 are nucleotide sequences encoding a heavy chain variable region and a light chain region of edrecolomab, respectively, and SEQ ID NOs: 72 and 76 are the amino acid sequences encoded by SEQ ID NOs: 71 and 75, respectively. SEQ ID NO: 77 is a nucleotide sequence encoding a mature CD16V sequence, and SEQ ID NO: 78 is the amino acid sequence encoded by SEQ ID NO: 77. SEQ ID NOs: 79, 81, 83, 85 and 87 are nucleotide sequences encoding sequences of CD16V mutants with introduced mutations, and SEQ ID NOs: 80, 82, 84, 86 and 88 are the amino acid sequences encoded by SEQ ID NOs: 79, 81, 83, 85 and 87, respectively. SEQ ID NO: 89 is a nucleotide sequence encoding an immature CD16V sequence, and SEQ ID NO: 90 is the amino acid sequence encoded by SEQ ID NO: 89. SEQ ID NO: 91 is the amino acid sequence of FLAG. The nucleotide sequences shown in SEQ ID NO: 92 is a nucleotide sequence encoding a fusion protein of the extracellular partial protein of the CD16V and signal domain of CD3ζ and CD137 via CD8a hinge and CD8 transmembrane domain, in which a signal sequence is linked to the N-terminus. The amino acid sequence shown in SEQ ID NO: 93 is the amino acid sequence encoded by SEQ ID NO: 92. The nucleotide sequences shown in SEQ ID NOs: 94, 96, 98, 100, and 102 are nucleotide sequences encoding a fusion protein of the extracellular partial protein of the CD16V in which a mutation has been introduced and signal domain of CD3ζ and CD137 via CD8a hinge and CD8 transmembrane domain, in which a signal sequence is linked to the N-terminus. The amino acid sequences shown in SEQ ID NOs: 95, 97, 99, 101, and 103 are amino acid sequences encoded by SEQ ID NOs: 94, 96, 98, 100, and 102, respectively. SEQ ID NO: 104 is an amino acid sequence of a signal sequence linked to the N-terminus of the CD16V CAR or CD16V mutant CAR proteins.

We claim:

1. A polypeptide comprising a modified Fc region of IgG, wherein the modified Fc region is non-naturally occurring and comprises at least one amino acid mutation compared to an Fc region of a wild type or naturally occurring IgG, and the polypeptide has essentially no binding activity to a wild type or naturally occurring Fcγ receptor and is capable of binding to a non-naturally occurring Fcγ receptor comprising at least one amino acid mutation compared to the wild type or naturally occurring Fcγ receptor.

2. The polypeptide according to claim 1, wherein the wild type or naturally occurring Fcγ receptor is a wild type or naturally occurring CD16A and said non-naturally occurring Fcγ receptor comprising at least one amino acid mutation is a non-naturally occurring CD16A comprising at least one amino acid mutation.

3. The polypeptide according to claim 2, wherein the wild type or naturally occurring CD16A comprises an amino acid sequence shown in SEQ ID NO: 78.

4. The polypeptide according to claim 2 or 3, wherein the CD16A comprising at least one amino acid mutation comprises at least one mutation selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K13 1E mutation).

5. The polypeptide according to claim 4, wherein the CD16A comprising at least one amino acid mutation comprises one or both of the K131D mutation and the K128E mutation.

6. The polypeptide according to claim 4, wherein the CD16A comprising at least one amino acid mutation comprises one or both of the K13 1E mutation and the K128E mutation.

7. The polypeptide according to claim 4, wherein the CD16A comprising at least one amino acid mutation comprises the K131D mutation, and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation).

8. The polypeptide according to claim 4, wherein the CD16A comprising at least one amino acid mutation comprises an amino acid sequence shown in SEQ ID NO: 80, SEQ ID NO: 82, SEQ ID NO: 84, SEQ ID NO: 86, or SEQ ID NO: 88.

9. The polypeptide according to any one of claims 1 to 8, wherein the polypeptide comprises a modified Fc region of human Igγ1, and the modified Fc region comprises (i) a mutation from a glutamic acid to an arginine at a position corresponding to position 269 according to EU index numbering (E269R mutation) and (ii) at least one mutation selected from (a) a glutamic acid to an arginine at a position corresponding to position 294 according to EU index numbering (E294R mutation) and (b) a glutamic acid to a lysine at a position corresponding to position 294 according to EU index numbering (E294K mutation).

10. The polypeptide according to any one of claims 1 to 9, wherein the polypeptide is an antibody.

11. The polypeptide according to claim 10, wherein the polypeptide is an antibody that binds to a cancer antigen.

12. A method of treating or preventing a disease or a disorder in a patient using immunotherapy, the method comprising administering to the patient a polypeptide according to any one of claims 1 to 11 and a cell expressing the non-naturally occurring Fcγ receptor comprising at least one amino acid mutation compared to a wild type or naturally occurring Fcγ receptor, wherein the polypeptide is capable of binding to said non-naturally occurring Fcγ receptor comprising at least one amino acid mutation.

13. The method according to claim 12, wherein the cell is a human immune cell.

14. The method according to claim 13, wherein the human immune cell is a cell selected from a T cell, macrophage, dendritic cell, NKT-cell, NK cell, microglia, osteoclast, granulocyte, monocyte, and innate immune cell.

15. The method according to any one of claims 12-14, wherein the cell is derived from a stem cell.

16. The method of claim 15, wherein the stem cell is selected from a pluripotent stem cell, hematopoietic stem cell, adult stem cell, fetal stem cell, mesenchymal stem cell, postpartum stem cell, multipotent stem cell, and embryonic germ cell.

17. The method of claim 16, wherein the stem cell is a pluripotent stem cell.

18. The method of claim 17, wherein the pluripotent stem cell is an induced pluripotent stem cell (iPS cell) or an embryonic stem cell (ES cell).

19. The method of any one of claims 12-18, wherein the cell comprises a genetically engineered disruption in a beta-2 microglobulin (B2M) gene.

20. The method of claim 19, wherein the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain.

21. The method of claim 20, wherein the HLA-1α chain is selected from HLA-A, HLA-B, HLA-C, HLA-E, HLA-F, and HLA-G.

22. The method of any one of claims 12-21, wherein the cell comprises a genetically engineered disruption in a human leukocyte antigen (HLA) class II-related gene.

23. The method of claim 22, wherein the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

24. The method of any one of claims 12-23, wherein the cell comprises one or more polynucleotides encoding a single chain fusion HLA class II protein or an HLA class II protein.

25. The method according to any one of claims 12 to 24, wherein the method is a method for treating or preventing cancer.

26. A pharmaceutical composition comprising a polypeptide according to any one of claims 1 to 11 and a pharmaceutically acceptable excipient.

27. The pharmaceutical composition according to claim 26 for combined use with a cell for immunotherapy, wherein the cell expresses a non-naturally occurring Fcγ receptor comprising at least one amino acid mutation compared to a wild type or naturally occurring Fcγ receptor, wherein the polypeptide is capable of binding to the non-naturally occurring Fcγ receptor comprising at least one amino acid mutation.

28. The pharmaceutical composition according to claim 27, wherein the cell is a human immune cell.

29. The pharmaceutical composition according to claim 28, wherein the human immune cell is a cell selected from a T cell, macrophage, dendritic cell, NKT-cell, NK cell, microglia, osteoclast, granulocyte, monocyte, and innate immune cell.

30. The pharmaceutical composition according to any one of claims 27-29, wherein the cell is derived from a stem cell.

31. The pharmaceutical composition of claim 30, wherein the stem cell is selected from a pluripotent stem cell, hematopoietic stem cell, adult stem cell, fetal stem cell, mesenchymal stem cell, postpartum stem cell, multipotent stem cell, and embryonic germ cell.

32. The pharmaceutical composition of claim 31, wherein the stem cell is a pluripotent stem cell.

33. The pharmaceutical composition of claim 32, wherein the pluripotent stem cell is an induced pluripotent stem cell (iPS cell) or an embryonic stem cell (ES cell).

34. The pharmaceutical composition of any one of claims 27-33, wherein the cell comprises a genetically engineered disruption in a beta-2 microglobulin (B2M) gene.

35. The pharmaceutical composition of claim 34, wherein the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain.

36. The pharmaceutical composition of claim 35, wherein the HLA-1α chain is selected from HLA-A, HLA-B, HLA-C, HLA-E, HLA-F, and HLA-G.

37. The pharmaceutical composition of any one of claims 27-36, wherein the cell comprises a genetically engineered disruption in a human leukocyte antigen (HLA) class II-related gene.

38. The pharmaceutical composition of claim 37, wherein the HLA class II-related gene is selected from regulatory factor X-associated ankyrin-containing protein (RFXANK), regulatory factor 5 (RFX5), regulatory factor X associated protein (RFXAP), class II transactivator (CITTA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

39. The pharmaceutical composition of any one of claims 27-38, wherein the cell comprises one or more polynucleotides encoding a single chain fusion HLA class II protein or an HLA class II protein.

40. A kit for treatment or prevention of a disease or disorder in a patient using immunotherapy, the kit comprising (i) a polypeptide according to any one of claims 1 to 11 and (ii) a cell expressing a non-naturally occurring Fcγ receptor comprising at least one amino acid mutation compared to a wild type or naturally occurring Fcγ0 receptor, wherein the polypeptide is capable of binding to the non-naturally occurring Fcγ receptor comprising at least one amino acid mutation.

41. The kit according to claim 40, wherein the cell is a human immune cell.

42. The kit according to claim 41, wherein the human immune cell is a cell selected from a T cell, macrophage, dendritic cell, NKT-cell, NK cell, microglia, osteoclast, granulocyte, monocyte, and innate immune cell.

43. The kit according to any one of claims 40-42, wherein the cell is derived from a stem cell.

44. The kit of claim 43, wherein the stem cell is selected from a pluripotent stem cell, hematopoietic stem cell, adult stem cell, fetal stem cell, mesenchymal stem cell, postpartum stem cell, multipotent stem cell, and embryonic germ cell.

45. The kit of claim 44, wherein the stem cell is a pluripotent stem cell.

46. The kit of claim 45, wherein the pluripotent stem cell is an induced pluripotent stem cell (iPS cell) or an embryonic stem cell (ES cell).

47. The kit of any one of claims 40-46, wherein the cell comprises a genetically engineered disruption in a beta-2 microglobulin (B2M) gene.

48. The kit of claim 47, wherein the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain.

49. The kit of claim 48, wherein the HLA-1α chain is selected from HLA-A, HLA-B, HLA-C, HLA-E, HLA-F, and HLA-G.

50. The kit of any one of claims 40-49, wherein the cell comprises a genetically engineered disruption in a human leukocyte antigen (HLA) class II-related gene.

51. The kit of claim 50, wherein the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

52. The kit of any one of claims 40-51, wherein the cell comprises one or more polynucleotides encoding a single chain fusion HLA class II protein or an HLA class II protein.

53. A cell expressing a non-naturally occurring CD16A comprising at least one amino acid mutation compared to a wild type or naturally occurring CD16A, wherein the at least one amino acid mutation is selected from (i) a lysine to an aspartic acid at a position corresponding to position 131 in SEQ ID NO: 78 (K131D mutation), (ii) a lysine to a glutamic acid at a position corresponding to position 128 in SEQ ID NO:
78 (K128E mutation), and (iii) a lysine to a glutamic acid at a position corresponding to position 131 in SEQ ID NO: 78 (K131E mutation), and wherein the non-naturally occurring CD16A comprises an amino acid sequence having 90% or more amino acid sequence identity with SEQ ID NO: 78.

54. The cell according to claim 53, wherein the CD16A comprising at least one amino acid mutation comprises one or both of the K131D mutation and the K128E mutation.

55. The cell according to claim 53, wherein the CD16A comprising at least one amino acid mutation comprises one or both of the K131E mutation and the K128E mutation.

56. The cell according to claim 53, wherein the CD16A comprising at least one amino acid mutation comprises the K131D mutation, and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation).

57. The cell according to claim 53, wherein the CD16A comprising at least one amino acid mutation comprises the amino acid sequence shown in SEQ ID NO: 80, SEQ ID NO: 82, SEQ ID NO: 84, SEQ ID NO: 86, or SEQ ID NO: 88.

58. The cell according to any of claims 53 to 57, wherein the cell is a human immune cell.

59. The cell according to claim 58, wherein the human immune cell is a cell selected from a T cell, macrophage, dendritic cell, NKT-cell, NK cell, microglia, osteoclast, granulocyte, monocyte, and innate immune cell.

60. The cell according to any one of claims 53-59, wherein the cell is derived from a stem cell.

61. The cell of claim 60, wherein the stem cell is selected from a pluripotent stem cell, hematopoietic stem cell, adult stem cell, fetal stem cell, mesenchymal stem cell, postpartum stem cell, multipotent stem cell, and embryonic germ cell.

62. The cell of claim 61, wherein the stem cell is a pluripotent stem cell.

63. The cell of claim 62, wherein the pluripotent stem cell is an induced pluripotent stem cell (iPS cell) or an embryonic stem cell (ES cell).

64. The cell of any one of claims 53-63, wherein the cell comprises a genetically engineered disruption in a beta-2 microglobulin (B2M) gene.

65. The cell of claim 64, wherein the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain.

66. The cell of claim 65, wherein the HLA-1α chain is selected from HLA-A, HLA-B, HLA-C, HLA-E, HLA-F, and HLA-G.

67. The cell of any one of claims 53-66, wherein the cell comprises a genetically engineered disruption in a human leukocyte antigen (HLA) class II-related gene.

68. The cell of claim 67, wherein the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB.

69. The cell of any one of claims 53-68, wherein the cell comprises one or more polynucleotides encoding a single chain fusion HLA class II protein or an HLA class II protein.

70. A pharmaceutical composition comprising a cell according to any one of claims 53 to 69 and a pharmaceutically acceptable excipient.

71. The pharmaceutical composition according to claim 70 for combined use with a polypeptide comprising a modified Fc region of IgG for immunotherapy, wherein the modified Fc region is non-naturally occurring and comprises at least one amino acid mutation compared to an Fc region of a wild type or naturally occurring IgG, and the polypeptide has essentially no binding activity to a wild type or a naturally occurring CD16A and is capable of binding to a non-naturally occurring CD16A comprising at least one amino acid mutation expressed by the cell.

72. The pharmaceutical composition according to claim 71, wherein the polypeptide comprises a modified Fc region of human Igγ1, and the modified Fc region comprises (i) a mutation from a glutamic acid to an arginine at a position corresponding to position 269 according to EU index numbering (E269R mutation) and (ii) at least one mutation selected from (a) a glutamic acid to an arginine at a position corresponding to position 294 according to EU index numbering (E294R mutation) and (b) a glutamic acid to a lysine at a position corresponding to position 294 according to EU index numbering (E294K mutation).

73. The pharmaceutical composition according to claim 71 or 72, wherein the polypeptide is an antibody.

74. The pharmaceutical composition according to claim 73, wherein the polypeptide is an antibody that binds to a cancer antigen.

75. The pharmaceutical composition according to any one of claims 71 to 74, wherein the pharmaceutical composition is for treating cancer.

76. A method for preparing a polypeptide comprising a modified Fc region of IgG, the method comprising the steps of:
1) providing polypeptides comprising a modified Fc region of IgG, wherein the modified Fc region is non-naturally occurring and comprises at least one amino acid mutation compared to a wild type or naturally occurring IgG;
2) measuring the binding activity of the polypeptides obtained in 1) to a wild type or naturally occurring Fcγ receptor;
3) measuring the binding activity of the polypeptides obtained in 1) to a non-naturally occurring Fcγ receptor comprising at least one amino acid mutation compared to a wild type or naturally occurring Fcγ receptor; and
4) selecting from the polypeptides obtained in 1) a polypeptide having essentially no binding activity to the wild type or naturally occurring Fcγ receptor and which binds to the non-naturally occurring Fcγ receptor comprising at least one amino acid mutation.

77. The method according to claim 76, wherein the wild type or naturally occurring Fcγ receptor is a wild type or naturally occurring CD16A and the non-naturally occurring Fcγ receptor comprising at least one amino acid mutation is a non-naturally occurring CD16A comprising at least one amino acid mutation.

78. The method according to claim 77, wherein the wild type or naturally occurring CD16A comprises the amino acid sequence shown in SEQ ID NO: 78.

79. The method according to claim 77 or 78, wherein the non-naturally occurring CD16A comprising at least one amino acid mutation comprises at least one mutation selected from (i) a lysine to an aspartic acid at a position corresponding to position 131 in SEQ ID NO: 78 (K131D mutation), (ii) a lysine to a glutamic acid at a position corresponding to position 128 in SEQ ID NO: 78 (K128E mutation), and (iii) a lysine to a glutamic acid at a position corresponding to position 131 in SEQ ID NO: 78 (K131E mutation).

80. The method according to claim 79, wherein the CD16A comprising at least one amino acid mutation comprises one or both of the K131D mutation and the K128E mutation.

81. The method according to claim 79, wherein the CD16A comprising at least one amino acid mutation contains one or both of the K131E mutation and the K128E mutation.

82. The method according to claim 79, wherein the CD16A comprising at least one amino acid mutation comprises the K131D mutation and further comprises at least one mutation selected from (iv) an asparagine to a glutamine at a position corresponding to position 38 in SEQ ID NO: 78 (N38Q mutation) and (v) an asparagine to a glutamine at a position corresponding to position 74 in SEQ ID NO: 78 (N74Q mutation).

83. The method according to any one of claims 76 to 82, wherein the non-naturally occurring polypeptide comprising a modified Fc region of IgG is an antibody.

84. The method according to claim 83, wherein the antibody is an antibody that binds to a cancer antigen.

85. The method according to claim 83 or 84, wherein the polypeptide comprising a modified Fc region of IgG is an antibody, and the method further comprises a step of contacting the antibody with an immune cell expressing the non-naturally occurring Fcγ receptor comprising at least one amino acid mutation and a cell expressing an antigen to which the antibody binds, and measuring antibody-dependent cellular cytotoxicity (ADCC) activity.

86. A method for preparing a non-naturally occurring Fcγ receptor, the method comprising the steps of:
1) providing non-naturally occurring Fcγ receptors comprising at least one amino acid mutation compared with a wild type or naturally occurring Fcγ receptor;
2) providing a polypeptide comprising an Fc region of wild type or naturally occurring IgG and a polypeptide comprising an Fc region of IgG comprising at least one amino acid mutation compared to the wild type or naturally occurring IgG;
3) measuring the binding activity of the non-naturally occurring Fcγ receptors obtained in 1) to the polypeptide comprising the Fc region of the wild type or naturally occurring IgG;
4) measuring the binding activity of the non-naturally occurring Fcγ receptors obtained in 1) to the polypeptide comprising the Fc region of IgG comprising at least one amino acid mutation; and
5) selecting from the non-naturally occurring Fcγ receptors obtained in 1) a non-naturally occurring Fcγ receptor having essentially no binding activity to the polypeptide comprising the Fc region of wild type or naturally occurring IgG and which binds to the polypeptide comprising the Fc region of the IgG comprising at least one amino acid mutation.

87. The method according to claim 86, wherein the Fcγ receptor is CD16A.

88. The method according to claim 87, wherein the wild type or naturally occurring Fcγ receptor is CD16A comprising the amino acid sequence shown in SEQ ID NO: 78.

89. The method according to any one of claims 86 to 88, wherein the Fc region of IgG comprising at least one amino acid mutation is an Fc region of human Igγ1 comprising at least one amino acid mutation compared to a wild type or naturally occurring human Igγ1 and comprises (a) a mutation from a glutamic acid to an arginine at a position corresponding to position 269 according to EU index numbering (E269R mutation) and (b) at least one mutation selected from (i) a mutation from a glutamic acid to an arginine at a position corresponding to position 294 according to EU index numbering (E294R mutation) and (ii) a mutation from a glutamic acid to a lysine at a position corresponding to position 294 according to EU index numbering (E294K mutation).

90. The method according to any one of claims 86 to 89, wherein the polypeptide comprising an Fc region of IgG comprising at least one amino acid mutation is an antibody.

91. The method according to claim 90, wherein the antibody is an antibody that binds to a cancer antigen.

92. The method according to claim 90 or 91, wherein the polypeptide comprising an Fc region of IgG comprising at least one amino acid mutation is an antibody, and the method further comprises a step of contacting the antibody comprising an Fc region of IgG comprising at least one amino acid mutation obtained in 2) with an immune cell expressing the Fcγ receptor comprising at least one amino acid mutation obtained in 1) and a cell expressing an antigen to which the antibody binds, and measuring antibody-dependent cellular cytotoxicity (ADCC) activity.

93. A method for preparing a binding pair comprising (a) a polypeptide comprising a modified Fc region of IgG and (b) a non-naturally occurring modified Fcγ receptor, the method comprising the steps of:
1) providing a polypeptide comprising an Fc region of wild type or naturally occurring IgG and polypeptides comprising a modified Fc region of IgG, wherein the modified Fc region is non-naturally occurring and comprises at least one amino acid mutation compared to the Fc region of the wild type or naturally occurring IgG;
2) providing a wild type or naturally occurring Fcγ receptor and non-naturally occurring modified Fcγ receptors, wherein the modified Fcγ receptor comprises at least one amino acid mutation compared to the wild type or naturally occurring Fcγ receptor;
3) measuring the binding activity of each Fcγ receptor obtained in 2) to each polypeptide obtained in 1); and
4) selecting (a) a polypeptide comprising a modified Fc region that binds to the modified Fcγ receptor and has essentially no binding activity to the wild type or naturally occurring Fcγ receptor, and (b) a modified Fcγ receptor that binds to the polypeptide comprising the modified Fc region and that does not bind to the Fc region of the wild type or naturally occurring IgG.

94. The method according to claim 93, wherein the Fcγ receptor is CD16A.

95. The method according to claim 93 or 94, wherein wild type or naturally occurring CD16A contains the amino acid sequence shown in SEQ ID NO: 78.

96. The method according to any one of claims 93 to 95, wherein the polypeptide comprising the modified Fc region of IgG is an antibody.

97. The method according to claim 96, wherein the antibody is an antibody that binds to a cancer antigen.

98. The method according to any one of claims 93-97, wherein the polypeptide comprising a modified Fc region of IgG selected in 4) is an antibody, and the method further comprises a step of contacting the antibody with an immune cell expressing the modified Fcγ receptor selected in 4) and a cell expressing an antigen to which the antibody binds, and measuring antibody-dependent cellular cytotoxicity (ADCC) activity.
